(19) United States
(12) Patent Application Publication
(10) Pub. No.: US 2017/0304500 A1
(43) Pub. Date: Oct. 26, 2017

(54) MICROFABRICATED TISSUE SCAFFOLDS AND METHODS OF MAKING AND USING THE SAME

(71) Applicant: TARA BIOSYSTEMS INC., New York, NY (US)

(72) Inventors: Miles Montgomery, Toronto (CA); Boyang Zhang, Toronto (CA); Milica Radisic, Toronto (CA)

(21) Appl. No.: 15/520,433

(22) PCT Filed: Oct. 20, 2015
(86) PCT No.: PCT/US2015/056501

§ 371 Date: Apr. 20, 2017

Related U.S. Application Data
(60) Provisional application No. 62/066,194, filed on Oct. 20, 2014.

(51) Int. Cl. A61L 27/38; A61L 27/14; A61L 27/00

(52) U.S. Cl. CPC A61L 27/3826; A61L 27/3834; A61L 27/14; A61L 27/00; A61L 2400/16

(57) ABSTRACT

The present description relates to the discovery of materials, devices, systems and methods for microfabrication of engineered tissue scaffolds for the growth and culture of biological tissues for tissue repair, transplantation, disease treatment, regenerative medicine, drug testing or combinations thereof. The engineered tissue scaffolds mimic native conditions and structures, including, e.g., native physiology, tissue architecture, vasculature, and other properties of native tissues.

### CROSS-REFERENCE TO RELATED APPLICATION

This application claims the benefit of U.S. Provisional Patent Application 62/066,194 filed Oct. 20, 2014 entitled “Microfabricated Tissue Scaffolds and Methods of Making and Using the Same,” which is incorporated herein by reference in its entirety for all purposes.

### BACKGROUND

### 1. Field of the Discovery

The present disclosure relates to microfabricated tissue scaffold devices for the culture, growth, and/or implantation of engineered tissues, including methods for making and using the same. The synthetic or engineered tissues may include, but are not limited to, cardiac, hepatic, neural, vascular, and muscle tissues. The methods, composition, and devices may be used in a variety of applications that include drug testing, tissue repair, tissue replacement, treatment, regenerative medicine or combinations thereof.

### 2. Background Information

Tissue engineering generally encompasses the use of biocompatible materials formed into a scaffold or structure for the culture and growth of cells and tissues. It is desirable to include the use of biochemical cues, e.g., growth factors, matrix proteins, etc. to improve, replace and/or mimic biological structures and/or functions. Tissue engineering is widely accepted as an interdisciplinary field that applies the principles of engineering and life sciences toward the development of biological substitutes that restore, maintain, or improve tissue function or a whole organ. Engineered tissue systems have significant potential in the area of regenerative medicine to restore and/or repair damage or diseased tissues (e.g., myocardial infarct), and have also been proposed for use in drug discovery and development as providing access to more accurate and physiologically relevant model systems for testing the pharmacokinetic and pharmacodynamic responses associated with pharmacologic agents.

Among the major challenges facing tissue engineering is the need for more complex and physiologically relevant engineered tissues that better mimic the structure, physiology, and function, of native tissues. Complex hierarchical cellular alignment is omnipresent in the human body, such as in blood vessels, neural networks, and cardiac or skeletal muscle. These structural features translate into critical functional characteristics. For instance, the highly organized and integrated pseudo-laminar myocardial syncytium correctly distributes an electrical propagation front that translates into orchestrated cardiac fiber contraction. The myocardium is also comprised of multiple cells types. The co-culture of multiple cell types has well-known to improve the functionality and survival of cardiac tissue in vitro and in vivo. Furthermore, the native myocardium contains sheets of fibroblast layers. Therefore, the ability to control the co-culture arrangement of engineered tissue constructs is a desirable feature. Traditional tissue culture methods such as embedding cells on foam scaffolds with a random pore distribution or a uniform hydrogel have been implemented to cast thick tissues rapidly, but often lack control over the intercellular organization required for organized tissue assembly.

In addition to the necessity of better mimicking native tissue structure and organization such that the engineered tissue is viable and physiologically functional, the implantable engineered tissue scaffold should also be chemically and mechanically stable, biocompatible and/or biodegradable, non-immunogenic, and/or elastic or deformable. Further still, the entire field is faced with a major limitation that prevents full adoption of tissue-engineered constructs: lab-grown functional tissue requires an invasive, surgical approach, to be placed in the body. If cells are simply injected with hydrogels in a minimally invasive manner, they do not possess tissue-level connections and high-level organization that are required for immediate functionality. The retention of the cells at the delivery site may also be compromised.

As such, improved engineered tissue systems are desired that address one or more of the above shortcomings. In particular, there is a need for engineered tissue systems that are: designed to mimic native tissue architecture, structurally robust but deformable, biocompatible and/or biodegradable, biologically functional, and able to be delivered in a minimally invasive matter. It is also desirable that such systems be readily adaptable for engineering a wide variety of tissue types, such as, e.g., cardiac, neural, vascular, musculoskeletal, gastrointestinal, etc. for use in, among other applications, drug testing, tissue repair, transplantation, disease treatment, regenerative medicine or combinations thereof.

### SUMMARY

The present description relates to the discovery of materials, devices, systems and methods for microfabrication and assembly of engineered tissue scaffolds, which are surprisingly and unexpectedly advantageous for the growth and culture of biological cells and/or tissues for, e.g., tissue repair, transplantation, disease treatment, regenerative medicine, drug testing or combinations thereof. In certain aspects the engineered tissue scaffolds mimic native conditions and structures, such as, but not limited to, native physiology, tissue architecture, geometry, vasculature, and other properties of native tissues.

In one aspect, the description provides engineered tissue scaffolds as described herein, which demonstrate shape memory (i.e., are reversibly deformable) making them adaptable to non-invasive methods of delivery, while, at the same time, are mechanically stable, functional, anisotropic, biocompatible and/or biodegradable.

In another aspect, the description provides engineered tissue scaffolds as described herein, which comprise polymer fiber layers that reversibly interlock or intercalate.

In still another aspect, the description provides engineered tissue scaffolds as described herein, which demonstrate shape memory (i.e., are reversibly deformable) as well as comprise polymer fiber layers that reversibly interlock or intercalate making them adaptable to non-invasive methods of delivery, while, at the same time, are mechanically stable, functional, anisotropic, biocompatible and/or biodegradable.

In certain embodiments, the description provides a tissue scaffold system comprising polymeric fibers, which are formulated and configured to allow the scaffold to be reversibly deformed. In certain embodiments, the “shape-memory” tissue scaffolds are conveniently deployed via, e.g., a catheter, in a minimally invasive procedure. In certain embodiments, the shape-memory tissue scaffold system comprises a network of micro- or nano-sized fibers or combination thereof, which form a polymer matrix or layer, wherein the fibers are arranged into a reversibly deformable design or geometrical configuration. In certain embodiments, the shape-memory tissue scaffold comprises a polymer matrix, wherein the polymer matrix includes an elastomeric polymer fibers. In certain embodiments, the elastomeric polymer fibers are configured into an array of rhomboid or bypyrimidal structures, e.g., diamond shapes.

In certain additional embodiments, the description provides a three-dimensional interlocking tissue scaffold or tissue scaffold system comprising a first polymer fiber layer having a top surface and a bottom surface and comprising an array of micro-hooks on at least one of the top or bottom surface. In certain embodiments, the first layer further comprises a second polymer fiber layer comprising loops or voids of sufficient size to allow intercalation or engagement with the micro-hooks of the first polymer fiber layer, wherein the two layers are reversibly attached or secured when physically abutted, overlaid or placed in apposition.

In certain embodiments, the description provides a multi-component, three-dimensional interlocking tissue scaffold system comprising a plurality of layers that are stacked so that they are at least partially overlapping with another layer, wherein at least every other polymer fiber layer in the stack comprises an array of micro-hooks on at least one surface. In a preferred embodiment, multiple layers are sequentially overlaid to construct a three-dimensional tissue scaffold of any desired thickness, and having internal channels or passageways allowing the growth and infiltration of cells. In an additional embodiment, at least one of the layers comprises a different type of cell from the other(s). In certain embodiments, each layer comprises a different type of cell seeded and cultured on the layer prior to being assembled such that the composite scaffold demonstrates a functional three-dimensional structure that functions approximately like native tissue.

In an additional aspect, the disclosure provides an interlocking tissue scaffold or system comprising a first polymer fiber mesh layer having fibers, which are formulated and configured to allow the scaffold to be reversibly deformed (i.e., a shape-memory scaffold as described herein), wherein the shape-memory polymer fiber mesh layer comprises micro-hooks on at least one surface. In certain additional embodiments, a multi-component interlocking tissue scaffold is provided comprising a first polymer fiber layer having fibers, which are formulated and configured to allow the scaffold to be reversibly deformed (i.e., a shape-memory scaffold as described herein), wherein the shape-memory polymer fiber mesh layer includes micro-hooks on at least one surface, and a second polymer fiber mesh layer comprising loops or voids of sufficient size to allow intercalation or engagement with the micro-hooks of the first polymer fiber layer, wherein the two layers are reversibly attached or secured when physically abutted, overlaid or placed in apposition.

In certain embodiments, the micro-hooks are polymeric structures formed on a surface of a polymer fiber layer as described herein. The micro-hooks may of any suitable size, shape, number and/or configuration sufficient to secure or affix the polymer fibers layers together. In a preferred aspect, the layers are reversibly affixed. For example, it should be understood that not all micro-hooks will be engaged by a loop on the abutting layer, however, a sufficient number should catch such that the layers are secured together. In a preferred embodiment, the micro-hooks are “T” shaped. In still other embodiments, the micro-hooks are comprised of polymer by securing a cross-bar onto a post structure extending approximately perpendicularly (relative to the x,y plane of the body of the scaffold, i.e., the z direction) from the top surface, bottom surface or both of the polymer fiber mesh layer. In certain embodiments, the micro-hooks are formed of poly(octamethylene maleate (anhydride) citrate) (PoMAC).

In any of the embodiments described herein, the polymer fiber tissue scaffold may be doped with additional micro- or nano-sized structures, which may serve as guides, supports or cues for tissue growth and maturation on the engineered tissue scaffold.

In any of the embodiments described herein, the scaffold polymer fibers comprise a polymer matrix comprising a suitable polymer material, including, for example, poly(dimethysiloxane (PDMS)), poly(methylmethacrylate (PMMA)), polystyrene, poly(glycerol sebacate), polyurethane, silk, metal. In certain embodiments, the polymer is a biodegradable polymer. The biodegradable polymer can be polylactic acid, poly(lactic-co-glycolic) acid, or poly(caprolactone), polyglycolide, polylactide, polyhydroxobutyrate, polyhydroxyalcanoic acids, chitosan, hyaluronic acid, hydrogels, poly(2-hydroxyethyl-methacrylate), poly(ethylene glycol), poly(L-lactide) (PLA), poly(octamethylene maleate (anhydride) acid), poly(octamethylene maleate (anhydride) citrate) (PoMAC). In certain embodiments, the polymer is a co-polymer comprising one or more of the above. In still additional embodiments, the scaffolds as described herein may include additional nanostructures such as, e.g., nanorods, posts or quantum dots. In a preferred embodiment, the polymer or co-polymer material is cross-linked, e.g., chemically or through UV light.

In any of the embodiments described herein, the matrix of the polymer fibers may include a bioadhesive component to facilitate securing the scaffold in place, in vivo, e.g., without the need or use of sutures. In certain embodiments, the bioadhesive is dopamine (3,4-dihydroxyphenethylamine). In certain embodiments, dopamine is coupled or covalently bound to a polymer subunit of the fiber polymer or co-polymer matrix.

In any of the embodiments described herein, the polymer fibers of the scaffold can be perfusable to allow exchange and/or passage of water and molecules, including proteins, drugs, nutrients, and metabolic waste materials. In certain other embodiments, perfusability may be implemented through the formation of pores in the scaffold polymer material, e.g., through the inclusion of porogens, e.g., poly(ethylene glycol) dimethyl ether (PEGDM). In still other embodiments, the scaffolds may be fabricated by any suitable means, including microfabrication, soft lithography processes (including, but not limited to step-and-flash imprint lithography (SFIL), 3D printing (i.e., additive manufacturing). molding, phase-shifting edge lithography, and nanoskiving).

In any of the embodiments described herein, the engineered tissue scaffold comprises cells that are seeded on or within the scaffold, which are then able to be grown, expanded, cultured, maintained, differentiated or a combination thereof. In certain embodiments, the cells to be seeded are precursor cells, e.g., stem cell-derived cardio myocytes, which are to be differentiated and expanded into at least one functional tissue cell type. In certain embodiments, the cells that are seeded are differentiated into a single tissue lineage. In additional embodiments, the cells are differentiated into two or more different tissues. In still additional embodiments, multiple cell types are seeded and co-cultured on or within the tissue scaffold. In still further embodiments, one or more of the different cell types are differentiated into tissues of different types on or within the tissue scaffold.

In certain embodiments, the cells used to grow the tissues on the scaffolds as described herein can be precursor or stem cells, including embryonic stem cells (“ESCs”), fetal stem cells (“FSCs”), and adult (or somatic) stem cells (“SSCs”). The stem cells, in terms of potency potential, can be totipotent (a.k.a. omnipotent) (stem cells that can differentiate into embryonic and extra-embryonic cell types), pluripotent stem cells (can differentiate into nearly all cells), multipotent stem cells (can differentiate into a number of cell types), oligopotent stem cells (can differentiate into only a few cell types), or unipotent cells (can produce only one cell type). Stem cells can be obtained commercially, or obtained/isolated directly from patients, or from any other suitable source. In various embodiments, the cells can be a cardiomyocyte, a hepatocyte, renal cell, chondrocyte, skin cell, contractile cell, blood cell, immune system cell, germ cell, neural cell, epithelial cell, hormone secreting cell, bone marrow cell, or a stem cell.

In any of the embodiments described herein, the engineered tissue scaffold polymer matrix comprises a sufficient or effective amount of a biochemical agent capable of promoting or modulating cell growth and differentiation. By way of non-limiting examples, the biochemical agent can comprise one or more growth factors, proteins or protein fragments, peptides, hormones, nucleic acids, antibodies, chemical activators or inhibitors of cell growth and/or differentiation or the like, which are known or become known to those of skill in the art.

In any of the embodiments described herein, the engineered tissue scaffold can further comprise an electrical cue, a physical or structural cue guide or combination thereof, to promote and/or modify the growth and/or orientation of one or more cell types. In certain embodiments, in particular wherein cardiac or other excitable cell or tissue type is grown on the scaffold, the cue is an electrical potential, e.g., electrical pulse, delivered across the cells growing on or within the tissue scaffold. In certain embodiments, the structural cue comprises a topographical feature that promotes the organized and/or directional growth of a cell or tissue. In a preferred embodiment, the scaffold fibers comprise a channel or a trough that extends contiguously, approximately coaxially along the length of the fiber. In certain embodiments, the fiber is configured to comprise a channel or trough that extends along the top and bottom surface of the polymeric fiber (e.g., in an “H” configuration) thereby allowing cell growth in both channels.

In any of the embodiments described herein, the engineered tissue scaffold additionally comprises an engineered tissue that is grown and cultured, or co-cultured on or within the scaffold.

In various other aspects, the present disclosure provides tissue scaffolds as described herein and methods for cultivating tissue thereon.

In still further aspects, the present disclosure also provides methods for fabrication and use of the tissue scaffold systems as described herein.

In various forms, the tissue scaffold systems of the disclosure comprises cardiac tissue, liver tissue, kidney tissue, cartilage tissue, skin, bone marrow tissue, or combinations of such tissues. In particular embodiments, the three-dimensional tissue system comprises cardiac tissue. In other particular embodiments, the three-dimensional tissue system comprises kidney tissue.

Where applicable or not specifically disclaimed, any one of the embodiments described herein are contemplated to be able to combine with any other one or more embodiments, even though the embodiments are described under different aspects of the invention.

The preceding general areas of utility are given by way of example only and are not intended to be limiting on the scope of the present disclosure and appended claims. Additional objects and advantages associated with the compositions, methods, and processes of the present invention will be appreciated by one of ordinary skill in the art in light of the instant claims, description, and examples. For example, the various aspects and embodiments of the invention may be utilized in numerous combinations, all of which are expressly contemplated by the present description. These additional advantages objects and embodiments are expressly included within the scope of the present invention. The publications and other materials used herein to illuminate the background of the invention, and in particular cases, to provide additional details respecting the practice, are incorporated by reference, and for convenience are listed in the appended bibliography.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated into and form a part of the specification, illustrate several embodiments of the present invention and, together with the description, serve to explain the principles of the invention. The drawings are only for the purpose of illustrating an embodiment of the invention and are not to be construed as limiting the invention. Further objects, features and advantages of the invention will become apparent from the following detailed description taken in conjunction with the accompanying figures showing illustrative embodiments of the invention, in which:

FIG. 1. Strategies for re-vascularization of damaged myocardium and vascularizing engineered cardiac tissues based on biological and engineering approaches. Depiction of current methods used to both directly vascularize native damaged myocardium (growth factor/gene therapy/microRNA, cell therapy, and bioactive scaffold) as well as to vascularize engineered cardiac tissue constructs (prevascularization, modular tissue assembly, guided tubulogenesis, and microfluidic blood vessels). Vascularization methods are listed in order of increasing applied engineered guidance. The dark arrows in the flow diagrams indicate the sequential steps involved in each strategy as well as the physical and biological effect generated with each strategy. The text box at the right end of the figure indicates the final step, which is also the final objective of each method.

FIG. 2. Illustration of A) Polycondensation and scaffold fabrication; B) Images showing the shape-memory of the scaffold before and after injection; C) Aligned cardiac tissue will be cultured under electrical stimulation; D) Scaffold made of a dopamine-bioadhesive scaffold attached to the tissue via: 1) Schiff base reaction with primary amine, 2) Michael addition with primary amine or 3) hydrogen bonding; E) Conceptual topography guided blood vessel growth.

FIG. 3. Illustration of experimental set-up for guided angiogenesis.

FIG. 4. Local concentration of autocrine growth factors. A simplified mathematical model of the VEGF concentration gradient in grooved and smooth samples, showing the effect of grooves on the local increase in the concentration of autocrine growth factors. (i and ii) Concentration profiles generated for different substrates. (i) Cross-sections showing a single groove (small rectangular region at the bottom) and the height of the culture medium on top of the substrate for each substrate. (ii) Cropped 100×200 μm close-up view showing the cross-section of a single groove for each substrate. The double-ended arrow indicates the width of the channel (25, 50, or 100 μm). The vertical dotted line indicates symmetry. The centrally positioned cell is shown as a black semicircle. The bottom of the image represents the top surface of the substrate for the flat substrate case. (iii) Horizontal concentration profile of VEGF along the bottom of the channel for grooved substrates and at the surface of the flat substrate, shown as relative VEGF concentrations with all values normalized to the value for flat substrate at the point (0, 0). In this steady-state model, VEGF was assumed to be secreted at a zero rate from a cell centrally positioned at the bottom of the groove or on a flat substrate.

FIG. 5. PoMaC patterned sheets with 50, 100, and 250 μm microchannels.

FIG. 6. Various shape-memory designs fabricated. Scale bar is 1 mm.

FIG. 7. Various shape-memory designs fabricated. Scale bar is 1 mm.

FIG. 8. Various shape-memory designs fabricated. Scale bar is 1 mm.

FIG. 9. Optimizing scaffold design. A) Four select designs that gave best injection results; B) Sample injection of a scaffold through a small 1 mm orifice shows the large change in shape-memory; C) Results of the injection success rate and the opening success rate reveal that design 4 is optimal.

FIG. 10. Mechanical properties of the final design when made of the adhesive polymer (ad), adhesive cross-linked polymer (adx), PoMaC, and the rat myocardium.

FIG. 11. Illustration of A) SEM image of a double-channeled scaffold cross-section. B) 500× magnification of a double-channeled fiber.

FIG. 12. Illustration of A) μCT images of a manually placed (control) and injected scaffold subcutaneously in an adult mouse; B) Quantifying μCT scans revealed that injected scaffolds re-open up 70% of the area compared to the control area (p-value 0.39); C) MicroCT image of a scaffold (highlighted in the white box) implanted subcutaneously in the dorsal region of an adult mouse.

FIG. 13. Engineered cardiac tissue. A) Fixed tissue sample on day 7 was stained for sarcomeric α-actinin and phalloidin, scale bar is 250 μm; B) Fluorescently labelled cardiac tissue was injected into the subcutaneous dorsal region of a sprague-dawley rat and immediately imaged in the far-red spectrum; C) No difference was seen when comparing the ET and MCR of cardiac sheets seeded on a diamond or oval patterned scaffold (independent t-test, ET p-value 0.79, MCR p-value=0.88, n=3); D) The presence of electrical stimulation during cell culture improved cardiac (independent t-test, ET p-value 0.16, MCR p-value 0.006 n=3); E) Improved alignment is seen in the stimulated group, structural staining for sarcomeric α-actinin (punctuate staining) and phalloidin, scale bar is 100 μm.

FIG. 14. Injected cardiac tissue. A) Live/dead staining of cells before and after injection shows minimal damage to the engineered cardiac tissue; B) The electrophysiological properties of the engineered cardiac tissue is not affected by the injection (paired t-test yielded an ET p-value=0.72 and a MCR p-value 0.21, n=6).

FIG. 15. A) Macroscopic view of the electrical stimulation bioreactor that holds the scaffold for cell culture. B) SEM image of the protruding features. C) Bright field image of a scaffold placed in the holder.

FIG. 16. A) Two-step polycondensation reaction scheme for producing a biodegradable dual cross-linkable bioadhesive polymer. In certain embodiments, citric acid is replaced with 1, 2, 4-butanetricarboxylic acid. B) ATR_FTIR spectra of PiCaB and PoMaC pre-polymers.

FIG. 17. ¹H-NMR spectra with the peaks labeled on top and the value of the integral below.

FIG. 18. Bioadhesive scaffold on rat myocardium. Still frames from movies comparing a how well a non-adhesive scaffold adheres to the surface of a rat heart as compared to an adhesive scaffold, before and after vigorous rinsing with PSB. This demonstrates that the adhesive scaffold adheres to the heart tissue better as the non-adhesive patch slides off easily.

FIG. 19. Illustrates A) A design that would have a cell-free patch surrounding the cells to provide bioadhesion; B) exemplary endoscopic tool that can directly place the patch on the desired location.

FIG. 20. Comparison of local concentrations between all cases. This is a close-up view of a 100 μm×100 μm square.

FIG. 21. One-step reaction scheme for PoMaC synthesis.

FIG. 22. Schematic of microfabrication methods for creating a scaffold with microarchitecture as described herein.

FIG. 23. Fabrication and physical characteristic of interlocking tissue scaffold system. (A) Comparing the hooks and loops of the conventional Velcro® system and(left), and the interlocking tissue scaffold design as described herein (right). (B) Illustration of the fabrication process of the interlocking tissue scaffold including a micro-injection step followed by the stamping step. (C) Illustration of seeding of cells on tissue scaffold and interlocking by stacking with second interlocking tissue scaffold layer. A Matrigel-based cell suspension is allowed to gel on the scaffold, and when removed from the tissue culture, substrate holes are formed. After self-assembly the compacted tissues can be handled and patterned; (D) SEM images revealing detailed interlocking tissue scaffold architecture with the T-shaped hooks and accordion mesh. Scale bar, 1 mm. Inset, high magnification SEM of T-shaped hooks. Scale bar, 500 μm. (E) SEM of the interlocks between individual interlocking tissue scaffold layers. Scale bar, 500 μm.

FIG. 24. Interlocking Tissue scaffold physical properties. (A) Representative force curve from the mechanical pull-off test of the tissue scaffold mesh (n=4). Inset scale bar, 5 mm. (B) Representative uniaxial tensile stress-strain plots of the interlocking tissue scaffold in the x direction (xD) and y direction (yD) (n−4). (C) Summary of the measured apparent modulus of the interlocking tissue scaffold in the x direction (xD), y direction (yD), and anisotropic ratio (xD/yD) (mean+/−SD, n=4). (D and E) Representative 3D renderings of profilometry data of the preassembled scaffold components: (D) Bottom mesh and post (n=3); (E) top hook (n=3). (F) Illustration of the cross-sectional view of an assembled scaffold labelled with measured heights (n=3).

FIG. 25. Characterization of cardiac cell growth on interlocking tissue scaffold. (A) Cardiac cell assembly around an interlocking tissue scaffold mesh over 7 days. Scale bar, 100 μm. (B) Area decrease (%) during 1-Hz paced contraction derived from scaffold deformation increased from day 4 to 6 (day 4: 0.9±0.3%; day 6: 1.4±0.07%, mean±SD, n=3). (C) Immunostaining of cardiac interlocking tissue scaffold on day 7 with sarcomeric α-actinin (bright) and F-actin (darker). (n=4). Scale bar, 30 μm. (D) SEM of an interlocking tissue scaffold showing tissue bundles (day 7); scale bar, 100 um. Inset, high-magnification SEM of a segment of interlocking tissue scaffold; scale bar, 100 um. (E) EC coating around 7-day-old cardiac tissue grew to confluence in 24 hours CD31, bright). Scale bar, 100 um. (F) CFDA cell tracker labelled endothelial cells; scale bar, 50 um. (G) Representative images of nuclear staining overlaid with nuclear orientation vectors along the long nuclear axis (n=3). Scale bar, 50 um. (H) Normalized distribution of orientation angles for cell nuclei and scaffold struts, respectively (representative trace of n=3).

FIG. 26. Tissue function and viability upon assembly and disassembly. (A) Co-culture conditions were instantaneously established in the z direction by assembling two-layers of tissue scaffold (day 7): one consisting of cardiac fibroblasts (FB) and the second comprising cardiomyocytes. Scale bar, 800 μm. Tissue interlocking was visualized with high-magnification fluorescent images focusing on layer 1 (L1) and layer 2 (L2). Scale bar, 200 μm. (B) Assembly of as interlocking tissue scaffold into a three-layer CM tissue construct. Scale bar, 800 μm High-magnification fluorescent images focused on L1 and L3 confirm interlocking between tissue scaffold layers. Scale bar, 200 μm. Arrowheads point to T-shaped micro-hooks protruding from the middle layer (L2) into the top layer (L1). (C) Electrical excitability parameters of the cardiac interlocking tissue scaffold (day 7) before assembly (mean±SD, n=8), after assembly (two-layer, mean±SD, n=4), after disassembly (mean±SD, n=8), and 1 day after disassembly (mean±SD, n=8). (D and E) Viability staining of CM interlocking tissue scaffold (day 4) (D) before (n=3) and (E) after the tissue assembly/disassembly process (n=4). Scale bar, 200 μm. Scaffold struts exhibit autofluorescence in the red channel. (F) Quantification of tissue viability from LDH activity in tissue culture media collected before (mean±SD, n=8) and after the tissue assembly/disassembly process (mean±SD, n=4)

FIG. 27. Patterned mosaic tissue assembly. (A to C) SEM of two cardiac tissues (day 4) assembled together and then cultured for an additional 3 days (n=4). (B and C) White arrows indicate locations where cells spread through a pathway created by the hood and loop configuration linking the two tissues together. Scale bars, 1 mm (A); 300 μm (B and C). (D and (E) Tissues (day 7) composed of cardiac FBs were labeled either green or red and arranged into (D) a 2D pattern (scale bar, 800 μm) and (E) an offset 2D pattern to extend the length of the construct (scale bar, 800 μm). (F) Two cardiac tissues (day 7) were labeled green or red and assembled together approximately at 45° angle. Scale bar, 800 μm

FIG. 28. Shape-memory (A) scaffold enables delivery and opening of a (B) cardiac patch from a pipette tip.

FIG. 29. Injection on top of rat heart

FIG. 30. A) In vivo cardiac tissue implantation studies. A) MicroCT on BaSO₄ stained scaffolds comparing patches surgically placed vs. injected subcutaneously in adult mice show a 30% decrease in opening area (n=3, p-value=0.039). B) Sub-cutaneous delivery of cardiac tissues in Lewis rats either placed surgically or injection histology analysis showed no statistical difference in CD31 or SMA staining after 7 days of implantation (n=3) C) Integration of cardiac patches placed onto healthy Lewis rat hearts with fibrin glue was seen after explanation at 7 days

FIG. 31. Porcine implantation pilot study A) Laparoscopic tool placement for minimally invasive cardiac patch delivery for accessing the left ventricle, pig (˜15 kg) were placed on their right side, black lines indicate rib location i) 5 mm trocar for tool access, ii) 5 mm endoscope, iii) 10 mm trocar for tool access B) Endoscopic camera views of stages of cardiac patch delivery on the left ventricle of epicardium i) cutting the pericardium, ii) Deploying the cardiac patch, iii) patch placed on left ventricle, iv) suturing patch to epicardium C) Representative live (green) dead (red) stains on cardiac tissue, positive control patches were left untouched in an incubator at 37° C. 5% CO₂, control patches were treated the same as the implants tissues except at the time of implantation were placed in PBS and put back into incubator for duration of implant (6 hours), implanted tissues were placed on the porcine epicardial surface for 6 hours after the chest was closed D) i-iv) SEM images of CO₂ critical point dried explant

FIG. 32. Base material physical properties under cell culture conditions. (A) Young's (n=4). (B) Material mass (mean±s.d, day 1, n=6, day 14 n=5).

FIG. 33. Hook and loop interlocking mechanism is a dominant factor governing the mechanical stability of the assembled two-layer structures. (B) Representative pull-off force plot indicated a gradual rise followed by a sharp drop in force as the scaffold was pulled off. (inset) Set-up with two scaffolds or tissues for pull-off force measurement. Bottom scaffold was anchored down with two micro-pins on one side of the scaffold. The two scaffolds were off-set to leave room for the micro-pins. Upper scaffold was pulled from the opposite side with another micro-pin attached to the Myograph. (B) Quantification of maximum pull-off force (mean±s.d) generated under three different scenarios indicates the presence of cells or short culture time (3 days) does not affect mechanical stability of the assembled structures. Cells(−) Culture time (−) represents pull-off force between two cell-free scaffolds (n=5), Cells(+) Culture time (−) represents pull-off force between two tissues (day 7) assembled immediately before pull-off test (n=4), Cells(+) Culture time (+) represents pull-off force between two tissues (day 4) assembled and then cultured for additional 3 days before the pull-off test (n=4). (C) Number of interlocking hooks counted prior to pull-off test (mean±s.d).

FIG. 34. Cardiac tissue contractility. (A) Quantification of axis shortening (%) during 1 Hz paced contraction on day 8 of culture (mean±s.d, n=4). (B) Illustration and a representative skeletonized trace of the scaffold struts with labels indicating the two directions of compression (xD, long edge direction and yD short edge direction).

FIG. 35. Immunostaining of cardiac Interlocking tissue scaffold on day 7 for sarcomeric α-actinin and F-actin at various locations of the tissues. Scale bar: 30 μm. Confocal sections were also shown individually to distinguish overlapping cells.

FIG. 36. Drug response. Spontaneously beating cardiac tissue (day 8) responding to stimulation with 300 nM epinephrine. EC50 for Epinephrine on rat cardiomyocytes were previously shown to range from 20 nM to 200 nM57. Increase in contraction rate is apparent.

FIG. 37. Co-culture of cardiac and endothelial cells. (A,B) Fluorescent image of Interlocking tissue scaffold stained with live and dead cell marker (CFDA, green and PI, red, scale bar: 200 μm). Scaffold struts exhibit autofluorescence in red. Tissues were first cultured in cardiomyocyte media for 4 days, then the tissues were coated (A) with or (B) without ECs and cultured for additional 4 days in EGM-2 media. Finally, the tissues were placed in 125 mL orbital shaker flasks at 160 RPM in 25 mL of EGM2 media for additional 3 days (n=4). Scale bar: 200 μm. (C,D) Quantification of the electrical excitability parameters at the end of the tissue culture (n=4).

FIG. 38. Scanning electron micrograph of the assembled two layer cardiac tissue cultivated for 3 days. Hooks from the bottom Interlocking tissue scaffold are locked onto the struts of the top Interlocking tissue scaffold, forming a bridge for cell spreading and tissue integration. Scale bars shown on images.

FIG. 39. Scanning electron micrograph of an additional Interlocking tissue scaffold design with spring-like structures that could potentially be used to enhance scaffold anisotropic mechanical properties and tissue anisotropic contraction. Scale bars shown on images.

### DETAILED DESCRIPTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

The present description relates to the discovery of materials, devices, systems and methods for microfabrication of engineered tissue scaffolds, which are surprisingly and unexpectedly advantageous for the growth and culture of biological tissues for tissue repair, transplantation, disease treatment, regenerative medicine, drug testing or combinations thereof. In certain aspects the engineered tissue scaffolds mimic native conditions and structures, such as, but not limited to, three-dimensional, native or native-like physiological function, tissue architecture, geometry, vasculature, and other properties of native tissues.

In addition, certain engineered tissue scaffolds as described herein demonstrate shape memory (i.e., are reversibly deformable) making them adaptable to non-invasive methods of delivery, while, at the same time, are mechanically stable, functional, anisotropic, biocompatible and/or biodegradable. In certain additional aspects, the disclosure provides modular, multi-layered, engineered tissue scaffolds which comprise, inter alia, structural features that allow the layers to be securely affixed to each other. In certain embodiments, the structures allow the layers to be reversibly affixed to each other. The laminar configuration allows for rapid assembly of multiple cell/tissue layer types creating a three-dimensional tissue architecture that closely mimics that of native tissue, e.g., fibroblast or endothelial tissue layers in apposition to organ cell types. The addition of growth and differentiation cues allows for the engineering of functional three-dimensional tissues that are directionally or anisotropically arranged.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The following terms are used to describe the present invention. In instances where a term is not specifically defined herein, that term is given an art-recognized meaning by those of ordinary skill applying that term in context to its use in describing the present invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the 10 United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects of the present invention, one or more of the present compounds described herein, are coadministered in combination with at least one additional bioactive agent. In particularly preferred aspects of the invention, the co-administration of compounds results in synergistic activity and/or therapy.

The term “treatment” as used herein includes any treatment of a condition or disease in an animal, particularly a mammal, more particularly a human, and includes: (i) preventing the disease or condition from occurring in a subject which may be predisposed to the disease but has not yet been diagnosed as having it; (ii) inhibiting the disease or condition, i.e. arresting its development; relieving the disease or condition, i.e. causing regression of the condition; or (iii) ameliorating or relieving the conditions caused by the disease, i.e. symptoms of the disease.

The term “effective” is used to describe an amount of a compound, composition or component which, when used within the context of its intended use, effects an intended result.

The term “therapeutically effective amount” refers to that amount which is sufficient to effect treatment, as defined herein, when administered to a mammal in need of such treatment. The therapeutically effective amount will vary depending on the subject and disease state being treated, the severity of the affliction and the manner of administration, and may be determined routinely by one of ordinary skill in the art.

It will be understood that, although the terms “first”, “second”, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of example embodiments.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

As used herein, the term “about” or “approximately” when preceding a number or number range means that a certain variance from the value is encompassed. The skilled artisan at the time of the present invention would appreciate that some minor variation is inherent in typical methods and measurements in the art, and therefore, the terms encompass routine, minor variation.

As used herein, the term “hydrogel” refers to a physically or chemically cross-linked polymer network that is able to absorb large amounts of water and is a common material for forming tissue engineering scaffolds. They can be classified into different categories depending on various parameters including the preparation method, the charge, and the mechanical and structural characteristics. Reference can be made to S. Van Vlierberghe et al., “Biopolymer-Based Hydrogels As Scaffolds for Tissue Engineering Applications: A Review,” Biomacromolecules, 2011, 12(5), pp. 1387-1408, which is incorporated herein by reference.

As used herein, unless the context indicates otherwise the term “microfabrication” is a concept that includes fabrication on a nanometer or micrometer level, including microfabrication and nanofabrication. Methods for microfabrication are well known in the art. Reference to certain microfabrication techniques that may be applicable in the invention include, for example, U.S. Pat. Nos. 8,715,436, 8,609,013, 8,445,324, 8,236,480, 8,003,300, as well as Introduction to Microfabrication (2004) by S. Franssila. ISBN 0-470-85106-6, each of which are incorporated herein by reference.

The term “microfabricated structure” as used herein is a concept that includes one or more structures occupying a two- or three-dimensional space, including a structure fabricated on a nanometer or micrometer scale. The term “two-dimensional” means on a surface in either vertical or horizontal space.

As used here, the term “PDMS” refers to the polymer poly(dimethylsiloxane). Polydimethylsiloxane (PDMS) belongs to a group of polymeric organosilicon compounds that are commonly referred to as silicones. PDMS is the most widely used silicon-based organic polymer, and is particularly known for its unusual rheological (or flow) properties. PDMS is optically clear, and, in general, inert, non-toxic, and non-flammable. It is also called dimethicone and is one of several types of silicone oil (polymerized siloxane).

As used herein, the term “POMac” refers to poly(octamethylene maleate (anhydride) citrate) (POMaC) or the POMac prepolymer which comprises a mixture of 1,8-octandiol, citrate acid, and maleic anhydride. Reference can be made to Tran et al., “Synthesis and characterization of a biodegradable elastomer featuring a dual crosslinking mechanism,” Soft Matter, Jan. 1, 2010; 6(11): 2449-2461, which is incorporated herein by reference in its entirety.

As used herein, the term “tunability” as it is used in reference to a “tunable” polymer, e.g., POMaC, refers to the capability of adjusting the process of polymerization of a polymer in a manner that allows for the formation of a resultant polymer product to have different mechanical and/or physical properties, such as elasticity, stiffness, and/or reactivity, or other properties. This concept is referred to in the context of certain polymers, such as POMac, that may be advantageously used in various embodiments/devices of the present invention, e.g., polymer wires, scaffolds, scaffold layers, and other components. Tunable polymers, such as POMaC, may have adjustable or “tunable” properties by adjusting, for example, (a) the degree or quantity of UV crosslinking or (b) the ratio of pre-polymer units that form the polymer, e.g., the ratio of polymer components, e.g., 1,8-octanediol, citric acid, and maleic anhydride in the case of POMac. In addition, certain embodiments, the polymer scaffolds are formed with pores of various sizes using porogens. The controlled formation of pores can also be regarded as an aspect of tunability, and in particular, pore size, distribution, and amount may be controlled as exemplified herein by the include of different amounts of polyethylene glycol dimethyle ether (PEGDME) or an equivalent during the UV crosslinking stage, wherein the PEGDME will “block” crosslinkages from forming, thereby, imparting pores of various pores.

In one aspect the disclosure provides a tissue scaffold or system comprising polymeric fibers, which are formulated and configured to allow the scaffold to be reversibly deformed. Such “shape-memory” tissue scaffolds are deployed in certain embodiments via, e.g., a catheter, in a minimally invasive procedure. In certain embodiments, the shape-memory tissue scaffold or system comprises a matrix including a network of micro- or nano-sized fibers or combination thereof, wherein the fibers are arranged into a reversibly deformable design or geometrical configuration. In certain embodiments, the fibers of the shape-memory tissue scaffold are comprised of polymer matrix, wherein the polymer matrix includes an elastomeric polymer fiber component. In certain embodiments, the elastomeric polymer fibers are configured into an array of rhomboid or bypyrimidal structures, e.g., diamond shapes. In certain additional embodiments, the deformable tissue scaffold as described herein comprises a deployed or “re-opened” area that is at least 10%, 20%, 30%, 40%, 50%, 60%, 70%, 80%, 90%, or 100% of the area of the scaffold prior to being deformed, e.g., compressed or folded.

In another aspect, the description provides engineered polymer fiber matrix layer having at least one surface comprising an array of micro-hooks on the at least one surface. The micro-hooks are suitable for intercalating, attaching or engaging additional polymer fiber layers when overlaid, at least partially. In a preferred embodiment, the micro-hooks allow for reversible securing of additional polymer fiber layers.

In another aspect, the description provides engineered three-dimensional tissue scaffolds as described herein, which comprise polymer fiber layers that reversibly interlock, intercalate or engage. In certain embodiments, the three-dimensional interlocking tissue scaffold comprises a first polymer fiber layer having a top surface and a bottom surface and comprising an array of micro-hooks on at least one surface. In certain embodiments, the scaffold further comprises another polymer fiber layer comprising loops or voids of sufficient size to allow intercalation or engagement with the micro-hooks of the first polymer fiber layer. In a preferred embodiment, the layers are reversibly secured or attached when physically abutted, overlaid or placed in apposition.

The three-dimensional tissue scaffolds as described herein can comprise any desired number of polymer fiber layers including. As such, in certain embodiments, the three-dimensional tissue scaffold comprises 2, 3, 4, 5, 6, 7, 8, 9, 10 or more layers.

The polymer fiber layers as described herein can be configured in any desired geometrical pattern or design. For example, in any of the embodiments described herein, the polymer fiber layers can be a woven or non-woven (e.g., cast) mesh of fibers. It is desirable that the selected polymer fiber design contain regular or irregular voids or pores to allow cell and tissue growth through the fiber layer, and around the fibers.

In certain embodiments, the disclosure provides a multi-component, three-dimensional tissue scaffold or system comprising a plurality of vertically assembled layers wherein the layers alternate between a first polymer fiber layer having micro-hooks, and second polymer fiber layer comprising loops or voids capable of reversibly engaging the micro-hooks of the abutting layer. In certain embodiments, the disclosure provides a multi-component, three-dimensional tissue scaffold or system comprising a plurality polymer fiber layers each having micro-hooks assembled such that at least a portion of each layer overlaps with the layer below it and wherein the micro-hooks intercalate or engage with fibers in another layer when placed in apposition, i.e., “sandwiched,” such that the layers are secured or affixed, e.g., reversibly secured or affixed. In certain embodiments, the edges of the fiber layers are aligned and arranged in a vertical stack such that one is approximately on top of the other.

In a preferred embodiment, multiple layers are sequentially overlaid to construct a three-dimensional tissue scaffold of any desired thickness, and having internal channels or passageways allowing the growth and infiltration of cells. In an additional embodiment, a three-dimensional tissue scaffold is provided as described herein, and comprising at least two polymer fiber layers wherein a different type of cell is seeded and cultured on each layer prior to being assembled such that the composite scaffold demonstrates a functional three-dimensional structure. In such a configuration, the scaffold more closely approximates the organization of native tissue.

In any of the aspects or embodiments described herein, the respective layers of the three-dimensional tissue scaffold system can have the same, similar, or completely different geometrical design, shape, thickness, chemical cues, physical features, etc. For example, when layers have the same designs are overlaid, the z axis will comprise fiber walls and channels that are contiguous and allow for unobstructed growth. In contrast, layers having disparate designs can be overlaid in order to effect the direction or ability of the tissue to grow in any particular direction. By applying the teachings as described herein, the skilled artisan will be able to select the appropriate combination of layer designs to better represent the native tissue environment.

In an additional aspect, the disclosure provides a tissue scaffold system comprising a first polymer fiber layer having fibers, which are formulated and configured to allow the scaffold to be reversibly deformed (i.e., a shape-memory scaffold as described herein), wherein the shape-memory polymer fiber mesh layer includes micro-hooks on at least one surface. In certain additional embodiments, a multi-component tissue scaffold is provided comprising a first polymer fiber layer having fibers, which are formulated and configured to allow the scaffold to be reversibly deformed (i.e., a shape-memory scaffold as described herein), wherein the shape-memory polymer fiber layer includes micro-hooks on at least one surface, and a second polymer fiber mesh layer comprising loops or voids of sufficient size to allow intercalation or engagement with the micro-hooks of the first polymer fiber layer, wherein the two layers are reversibly secured or affixed when physically overlaid or placed in apposition. In certain embodiments, the scaffold comprises multiple polymer fiber layers, wherein each layer includes micro-hooks such that each layer is reversibly secured or affixed to the next (i.e., the layer above and/or below it).

In certain embodiments, the micro-hooks are polymeric structures formed on a surface of a polymer fiber layer as described herein. The micro-hooks may of any suitable size, shape, number and/or configuration sufficient to secure or affix the polymer layers together. In a preferred aspect, the layers are reversibly affixed. For example, it should be understood that not all micro-hooks will be engaged by a fiber loop on an abutting layer, however, a sufficient number should catch such that the layers are secured together. Significantly, the micro-hooks also allow for the layers to be separated with a force that is low enough to preserve the cells and tissue growing thereon. In a preferred embodiment, the micro-hooks are “T” shaped. In still other embodiments, the micro-hooks are comprised of a polymer by securing a cross-bar onto a post structure extending approximately perpendicularly (relative to the x,y plane of the body of the scaffold, i.e., the z direction) from the top surface, bottom surface or both of the polymer fiber layer. In certain embodiments, the micro-hooks are formed of poly(octamethylene maleate (anhydride) citrate) (PoMAC).

In any of the embodiments described herein, the polymer fiber tissue scaffold may be doped with additional micro- or nano-sized structures, which may serve as guides, supports or cues for tissue growth and maturation on the engineered tissue scaffold.

In still additional embodiments, the polymer fiber can be doped with a biologically active agent, for example, a cellular growth factor or inhibitor, a drug, a cytotoxic agent, etc.

In any of the embodiments described herein, the scaffold polymer fibers comprise a polymer matrix comprising a suitable polymer material, including, for example, poly(dimethysiloxane (PDMS)), poly(methylmethacrylate (PMMA)), polystyrene, poly(glycerol sebacate), polyurethane, silk, metal. In certain embodiments, the polymer is a biodegradable polymer. The biodegradable polymer can be polylactic acid, poly(lactic-co-glycolic) acid, or poly(caprolactone), polyglycolide, polylactide, polyhydroxobutyrate, polyhydroxyalcanoic acids, chitosan, hyaluronic acid, hydrogels, poly(2-hydroxyethyl-methacrylate), poly(ethylene glycol), poly(L-lactide) (PLA), poly(octamethylene maleate (anhydride) acid), poly(octamethylene maleate (anhydride) citrate) (PoMAC). In certain embodiments, the polymer is a co-polymer comprising one or more of the above. In still additional embodiments, the scaffolds as described herein may include additional nanostructures such as, e.g., nanorods, posts or quantum dots. In a preferred embodiment, the polymer or co-polymer material is cross-linked, e.g., chemically or through UV light.

In any of the embodiments described herein, the polymer fiber matrix may include a bioadhesive component to facilitate securing the scaffold in place, in vivo, e.g., without the need or use of sutures. In certain embodiments, the bioadhesive is dopamine (3,4-dihydroxyphenethylamine). In certain embodiments, dopamine is coupled or covalently bound to a polymer subunit of the fiber polymer or co-polymer matrix.

In any of the embodiments described herein, the polymer fibers or fiber layers of the scaffold can be perfusable to allow exchange and/or passage of water and molecules, including proteins, drugs, nutrients, and metabolic waste materials. In certain other embodiments, perfusability may be implemented through the formation of pores in the scaffold polymer material, e.g., through the inclusion of porogens, e.g., poly(ethylene glycol) dimethyl ether (PEGDM). In still other embodiments, the scaffolds may be fabricated by any suitable means, including microfabrication, soft lithography processes (including, but not limited to step-and-flash imprint lithography (SFIL), 3D printing (i.e., additive manufacturing). molding, phase-shifting edge lithography, and nanoskiving).

In any of the embodiments described herein, the engineered tissue scaffold comprises cells that are seeded on or within the scaffold, which are then able to be grown, expanded, cultured, maintained, differentiated or a combination thereof. In certain embodiments, the cells to be seeded are precursor cells, e.g., stem cell-derived cardiac myocytes, which are to be differentiated and expanded into at least one functional tissue cell type. In certain embodiments, the cells that are seeded are differentiated into a single tissue lineage. In additional embodiments, the cells are differentiated into two or more different tissues. In still additional embodiments, multiple cell types are seeded and co-cultured on or within the tissue scaffold. In still further embodiments, one or more of the different cell types are differentiated into tissues of different types on or within the tissue scaffold.

In certain embodiments, the cells used to grow the three-dimensional tissues of the invention can be stem cells, including embryonic stem cells (“ESCs”), fetal stem cells (“FSCs”), and adult (or somatic) stem cells (“SSCs”). The stem cells, in terms of potency potential, can be totipotent (a.k.a. omnipotent) (stem cells that can differentiate into embryonic and extra-embryonic cell types), pluripotent stem cells (can differentiate into nearly all cells), multipotent stem cells (can differentiate into a number of cell types), oligopotent stem cells (can differentiate into only a few cell types), or unipotent cells (can produce only one cell type). Stem cells can be obtained commercially, or obtained/isolated directly from patients, or from any other suitable source.

In various embodiments, the cells can be a cardiomyocyte, a hepatocyte, renal cell, chondrocyte, skin cell, contractile cell, blood cell, immune system cell, germ cell, neural cell, epithelial cell, hormone secreting cell, bone marrow cell, or a stem cell.

In any of the embodiments described herein, the cells to be seeded on a tissue scaffold as described here can be seeded in a hydrogel, e.g., collagen gel, optionally comprising additional proteins, proteoglycans, polysaccharides, or extracellular matrix factors in order to promote growth and attachment of the seeded cells to the tissue scaffold.

In any of the embodiments described herein, the engineered tissue scaffold polymer matrix comprises a sufficient or effective amount of a biochemical agent capable of promoting or modulating cell growth and differentiation. By way of non-limiting examples, the biochemical agent can comprise one or more growth factors, proteins or protein fragments, peptides, hormones, nucleic acids, antibodies, chemical activators or inhibitors of cell growth and/or differentiation or the like, which are known or become known to those of skill in the art.

In any of the embodiments described herein, the engineered tissue scaffold can further comprise an electrical cue, a physical or structural cue guide or combination thereof, to promote and/or modify the growth and/or orientation of one or more cell types. In certain embodiments, in particular wherein cardiac or other excitable cell or tissue type is grown on the scaffold, the cue is an electrical potential, e.g., electrical pulse, delivered across the cells growing on or within the tissue scaffold. In certain embodiments, the electrical cue can be delivered via the use of certain piezoelectric polymers that generate electrical fields when deformed. In certain embodiments, the structural cue comprises a topographical feature that promotes the organized and/or directional growth of a cell or tissue.

In a preferred embodiment, the scaffold fibers comprise a channel or a trough that extends contiguously, approximately coaxially along the length of the fiber. In certain embodiments, the fiber is configured to comprise a channel or trough that extends along the top and bottom surface of the polymeric fiber (e.g., in an “H” configuration) thereby allowing cell growth in both channels. Channels such as those described above are advantageous for the growth of, e.g., endothelial cells, and promote the formation of micro vessels throughout the fiber scaffold.

In any of the embodiments described herein, the engineered tissue scaffold additionally comprises an engineered tissue that is grown and cultured, or co-cultured on or within the scaffold.

In various other aspects, the present disclosure provides devices and methods for cultivating tissue.

In still further aspects, the present disclosure also provides methods for fabrication and use of the tissue scaffold systems as described herein.

In various forms, the three-dimensional tissue system of the disclosure comprises cardiac tissue, liver tissue, kidney tissue, cartilage tissue, skin, bone marrow tissue, or combinations of such tissues. In particular embodiments, the three-dimensional tissue system comprises cardiac tissue. In other particular embodiments, the three-dimensional tissue system comprises kidney tissue.

Where applicable or not specifically disclaimed, any one of the embodiments described herein are contemplated to be able to combine with any other one or more embodiments, even though the embodiments are described under different aspects of the invention.

In certain additional aspects, the disclosure provides engineered tissue scaffolds as described herein, wherein the polymer fiber matrix is configured for the controlled release of a biochemical or pharmaceutical agent. By “controlled release” it is meant for purposes of the present invention that therapeutically active compound is released from the preparation at a controlled rate or at a specific site, for example, the intestine, or both such that therapeutically beneficial blood levels (but below toxic levels) are maintained over an extended period of time, e.g., providing a 12 hour or a 24 hour dosage form.

The skilled artisan will appreciate that reference can be made to resources available in the state of the art regarding the making and use of tissue engineering scaffolds and, in particular, reference case be made to the scaffold materials described in Dhandayuthapani et al., “Polymeric Scaffolds in Tissue Engineering Application: A Review; International Journal of Polymer Science, Vol. 2011 (2011), pages 1-19, which is incorporated herein by reference.

In addition, the tissue scaffold systems as described herein may also use semi-synthetic materials, such as those disclosed in Rosso et al., “Smart materials as scaffolds for tissue engineering,” J Cell Physiol. 2006 December; 209(3):1054. Such scaffolds may contain oligopeptide cleaving sequences specific for matrix metalloproteinases (MMPs), integrin binding domains, growth factors, anti-thrombin sequences, plasmin degradation sites, and morphogenetic proteins. Such semi-synthetic materials aim to confer “intelligent” semi-synthetic biomaterials, having advantages offered by both the synthetic materials (e.g., processability, mechanical strength) and by the natural materials (e.g., specific cell recognition, cellular invasion, and the ability to supply differentiation/proliferation signals). Due to their characteristics, these semi-synthetic biomaterials represent a new and versatile class of biomimetic hybrid materials that hold clinical promise in serving as a source of materials for the scaffolds described herein.

The surface of the scaffolds as described herein may also be modified with any suitable surface treatments, including chemical modifications (such as, for example, ligands, charged substances, bind agents, growth factors, antibiotics, antifungal agents), or physical modifications (such as, for example, spikes, curved portions, folds, pores, uneven portions, or various shapes and topographies) which may facilitate the tissue culture process.

In various embodiments, the cells that may be seeded and cultivated in the tissue scaffold systems disclosed herein may include, but are not limited to, cardiac cells, liver cells, kidney cells, cartilage cells, skin cells, bone marrow cells, or combinations of such tissues. In particular embodiments, the tissue scaffold systems disclosed herein are suitable for growing cardiac tissue, hepatic tissue, or kidney tissue. In certain embodiments, the tissues formed in or on the systems described herein are three-dimensional tissues.

In various other embodiments of the, the tissue scaffold systems disclosed herein may be seeded with stem cells or otherwise progenitor cells which are capable of developing into mature tissue types, e.g., mature cardiac, hepatic, or kidney tissue. Stem cells may include, but are not limited to embryonic stem cells and adult stem cells. In addition, stem cells contemplated for use with the herein described devices may have any degree of potency, including totipotent/omnipotent cells, pluripotent cells, multipotent cells, oligopotent cells, or unipotent cells (e.g., progenitor cells).

In embodiments involving cardiac cells (or other electrically-stimulated cells), the tissue scaffold systems described herein can be further configured to include electrodes configured to generate an electric field across the scaffold system to promote growth and differentiation while culture the tissue in vitro. The direction of the electric field can be in any direction, but preferably in a direction that is generally parallel to the longitudinal axis. However, the orientation of the electric field is not limited and the positioning of the electrodes can be in any suitable format such that a suitable electric field can be generated. In certain embodiments, e.g., cardiac cells, the electric field facilitates that maturation of the cells to form tissue that more closely mimics the physiological and electrical properties of actual tissue, e.g., cardiac tissue.

In various embodiments, the tissue scaffolds as described herein can comprise any suitable material or combination of materials, which can include natural materials, such as collagen and collagen derivatives, natural suture material (e.g., animal intestines), cellulose and cellulose derivatives, proteoglycans, heparin sulfate, chondroitin sulfate, keratin sulfates, hyaluronic acid, elastin, fibronectin, and lamanin, etc., as well as synthetic materials, including various polymers and nanomaterials. Such choices can be based on a variety of parameters, which can include their material chemistry, molecular weight, solubility, shape and structure, hydrophilicity/hydrophobicity, lubricity, surface energy, water absorption degradation, and erosion mechanism.

The term “rate controlling polymer” as used herein includes hydrophilic polymers, hydrophobic polymers or mixtures of hydrophilic and/or hydrophobic polymers that are capable of retarding the release of the compounds in vivo. It is within the skill of those in the art to modify the control release polymer permeability, and dissolution characteristics to provide the desired controlled release profile (e.g., drug release rate and locus) without undue experimentation.

The controlled release polymer may comprise a hydrogel matrix, for instance, HPMC, or mixture of polymers which when wet will swell to form a hydrogel. The rate of release is controlled both by diffusion from the swollen mass, the erosion of the surface over time, and viscosities of the polymers used. Examples of suitable controlled release polymers to be used in this invention include hydroxyalkylcellulose, such as hydroxypropylcellulose and hydroxypropylmethyl-cellulose; poly(ethylene)oxide; alkylcellulose such as ethylcellulose and methylcellulose; carboxymethylcellulose; hydrophilic cellulose derivatives; polyethylene glycol; polyvinylpyrrolidone; cellulose acetate; cellulose acetate butyrate; cellulose acetate phthalate; cellulose acetate trimellitate; polyvinylacetate phthalate; hydroxypropylmethylcellulose phthalate; hydroxypropylmethylcellulose acetate succinate; poly(alkyl methacrylate); and poly (vinyl acetate). Other suitable hydrophobic polymers include polymers or copolymers derived from acrylic or methacrylic acid esters, copolymers of acrylic and methacrylic acid esters, zein, waxes, shellac and hydrogenated vegetable oils.

Methods

In another aspect the disclosure provides methods for treatment or amelioration of a disease state or disorder. For example, the engineered tissue scaffolds can be administered prophylactically or therapeutically to a subject in need thereof, wherein the tissue scaffold is effective for treating, preventing or ameliorating the effects of the disease or disorder.

In certain embodiments, the methods include a method of treating a disease or condition comprising providing a tissue scaffold as described herein, seeding a cell and culturing tissue growth on the scaffold, optionally implanting or contacting the scaffold having a cultured tissue thereon to a site in or on a subject in need thereof, wherein the engineered tissue scaffold is effective for treating or ameliorating at least one symptom of the disease or condition.

Identifying a subject in need of such treatment can be in the judgment of the subject or a health care professional and can be subjective (e.g., opinion) or objective (e.g., measurable by a test or diagnostic method). The therapeutic methods of the invention, which include prophylactic treatment, in general comprise administration of a tissue scaffold as described herein, such as a scaffold comprising a viable tissue grown thereon, to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like).

Diseases or disorders which can be treated, prevented or ameliorated via the tissue scaffolds as described herein may include, e.g., myocardial infarction, neurodegeneration, wound healing, among others. Additional exemplary diseases, disorders or conditions which may be treated include, but are not limited to burns, rheumatoid arthritis, osteoarthritis, juvenile chronic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, atopic dermatitis, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjodgren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), insulin-dependent diabetes mellitus, sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis and vitiligo. The human antibodies, and antibody portions of the invention can be used to treat autoimmune diseases, in particular those associated with inflammation, including, rheumatoid spondylitis, allergy, autoimmune diabetes, autoimmune uveitis.

In another aspect, the present description provides methods of making a three-dimensional tissue scaffold of the invention. In certain embodiments, the method comprises the steps of providing one or more polymer fiber layers as described herein, wherein the fibers form a matrix. In certain embodiments, the method comprises the steps of seeding and culturing a cell on the polymer fiber matrix.

In a preferred embodiment, the description provides a method of forming a polymer comprising performing polycondensation reaction including 1,8-octanediol, maleic anhydride, and an acid. In certain embodiments, the acid is at least one of 1,2,4-butanetricarboxylate, citric acid or a combination of both. In certain embodiments, the reaction is heated to about 160° C. until a clear solution is formed. In certain embodiments, the solution is cooled to about 140° C. for about 3 hours under nitrogen purge to form a pre-polymer. In certain additional embodiments, the prepolymer is dissolved in a solvent, e.g., 1, 4-dioxane, and purified. In additional embodiments, the polymer is purified by drop-precipitation into deionized water and lyophilized. In certain embodiments, the polymer is lyophilized for about 3 days.

In still additional embodiments, the purified pre-polymer solution is then mixed with a porogen. In certain embodiments, the porogen is poly(ethylene glycol) dimethyl ether (PEGDM, Mw˜500, Sigma). In still additional embodiments, the porogen is admixed at about 60 wt %. in certain embodiments, the pre-polymer/porogen solution further includes a UV-imitator. In certain embodiments, the UV-initiator is present in an about of about 5 wt %. In still additional embodiments, the UV initiator comprises 2-hydroxy-1-[4(hydroxyethoxy)phenyl]-2-methyl-1 propanone (Irgacure 2959).

In an additional embodiment, the polymeric material is injected into a mold or cast configured to comprise a network of channels. Following injection, the polymer is allowed to polymerize. In certain embodiments, the polymerized polymer is exposed to UV light. In a preferred embodiment, the UV light is delivered at about 16 mW/cm2 for about 3 minutes forming a polymer fiber matrix layer. In certain embodiments, polymer “posts” that extend vertically are annealed to the polymer fiber matrix layer. In still additional embodiments, polymer fiber cross-bars are annealed perpendicularly to the vertical posts (i.e., parallel to the plane of the polymer fiber matrix layer) thereby forming micro-hooks on the polymer fiber matrix layer.

In certain embodiments, the method includes layering plurality of polymer fiber layers vertically to form a three-dimensional tissue scaffold. In certain embodiments, the three-dimensional tissue scaffold comprises a plurality of alternating layers, wherein the layers are formed of a first polymer fiber layer including micro-hooks and a second polymer fiber layer having holes, loops or voids therethrough that are of sufficient size to allow intercalation or engagement with the micro-hooks of the first polymer fiber mesh layer. In certain embodiments, the method comprises the step of seeding and culturing a cell on the three-dimensional tissue scaffold.

The disclosure provides an exemplary functional shape-memory tissue scaffold as described herein that can be delivered in a minimally invasive manner. This tissue scaffold possesses the capability of deforming to fit through a small diameter needle and substantially regaining its original shape once injected. The reversibly deformable or elastic tissue scaffolds as described herein are particularly useful for applications with contractile tissues, e.g., muscle.

For example, the elastic tissue scaffolds as described herein can be placed over an ischemic region of the heart to remuscularize and revascularize to reduce overall damage. An exemplary design has been identified that has both shape-memory and anisotropic mechanical properties matching the myocardium. Cardiomyocytes have successfully been cultured on the scaffolds and injected through a 1 mm orifice with minimal tissue damage. In addition, a photocrosslinkable bioadhesive scaffold material is described that can be used to adhere a patch onto the surface of a heart.

Myocardial infarction (MI), commonly known as a heart attack, results from an insufficient blood supply to the heart which causes, on average, the death of 1 billion cells. The heart is unable to regenerate this damaged tissue, but implanting engineered heart tissue could potentially restore heart function. For example, implanted engineered cardiac tissues can be functionally integrated with the host heart and improve heart condition post-MI. However, typical lab-grown functional tissue requires an invasive, surgical approach, to be placed in the body.

A wide range of cell injection strategies to circumvent this invasive procedure have been proposed. Despite showing some improvements in cardiac function, these strategies have been plagued by excessive cell death after delivery, and minimal functional host integration. Applying biomaterials to the myocardium (the heart muscle) have been shown to reduce adverse changes to the heart's shape (cardiac remodeling) post-MI but are not long-term healing solutions. To improve cell retention and survival, cells have been delivered in a solution that solidifies when placed at the desired location. If cells are simply injected with hydrogels in a minimally invasive manner, they do not possess tissue-level connections and high-level organization that are required for immediate functionality. Thus, such an approach might only be practical for tissues that do not require the cells to have high-level organization and immediate function (e.g. cartilage). Heart repair requires an engineered tissue implant that provides functionality immediately.

Described herein are injectable, yet fully functional engineered tissue constructs. Flexible biodegradable shape-memory scaffolds can be used to culture functional engineered tissue, e.g., cardiac tissue. This exemplary scaffold's shape-memory will allow the tissue to collapse during injection, and subsequently regain its original shape once deployed in situ, while maintaining cell viability and tissue function.

Tissue vascularization is one of the greatest challenges in tissue engineering, especially in cardiac tissue engineering. The high metabolic rate of cardiomyocytes (CMs) is reflected by the capillary density in the heart; almost every CM neighbors a capillary to facilitate efficient mass transfer. Initial vascularization solutions stemmed from biological methods (i.e. growth factor delivery, gene therapy, cell therapy, etc.), which stimulate endogenous blood vessels to grow into the infracted myocardium, reducing the expansion of the infarct and improving heart function but with limited efficacy.

According to the current state of the art, tissue engineering methods placing a functional patch onto the heart requires opening of the chest. In clinical use, this would expose a patient to a significant risk, increase the recovery time and limit the usefulness of the patch-approach to those patients who would require an open heart surgery anyways, for example, those undergoing coronary artery bypass grafting. For these reasons, cells alone or hydrogels have met with little success. Engineered cardiac patches have been applied to the ischemic rat heart. Despite the positive results of simultaneous remuscularization and protective paracrine signaling, it is still difficult to fully leverage the potential of engineered cardiac patches due to challenges in achieving adequate vascularization, vascular integration, and tissue engraftment hence impeding progress towards clinical translation. Vascularization is crucial in two respects: 1) without a functional and mature pre-vascular network in vitro, relatively thick (≧1 cm) physiologically relevant three-dimensional (3D) cardiac tissue cannot be cultivated and 2) successful integration with the host will depend on rapid initial vascular anastomosis and long-term integration with the host vasculature.

Cardiac tissue is utilized as a model system because: a) cardiomyocytes are extremely sensitive cells, b) immediate functionality of the heart tissue is desired, and c) this functionality can be easily assessed in vitro through measurements of contractile properties. The anisotropic (directionally dependent) stiffness of heart tissue is an important design parameter that the scaffold mechanical properties should replicate. Furthermore, ensuring that the material degrades at the same rate as the heart heals is an important feature for a successful cardiac tissue implant. Combining these requirements in conjunction with minimally invasive tissue delivery is a complex multidisciplinary engineering challenge.

### EXAMPLES

It is understood that the examples and embodiments described herein are for illustrative purposes only and that various substitutions, modifications or changes in light thereof will be suggested to persons skilled in the art and are included within the spirit and purview of this application and are considered within the scope of the appended claims. The following examples are given by way of example of the preferred embodiments, and are in no way considered to be limiting to the invention. For example, the relative quantities of the ingredients may be varied to achieve different desired effects, additional ingredients may be added, and/or similar ingredients may be substituted for one or more of the ingredients described. All publications, patents, and patent applications cited herein are hereby incorporated by reference in their entirety for all purposes.

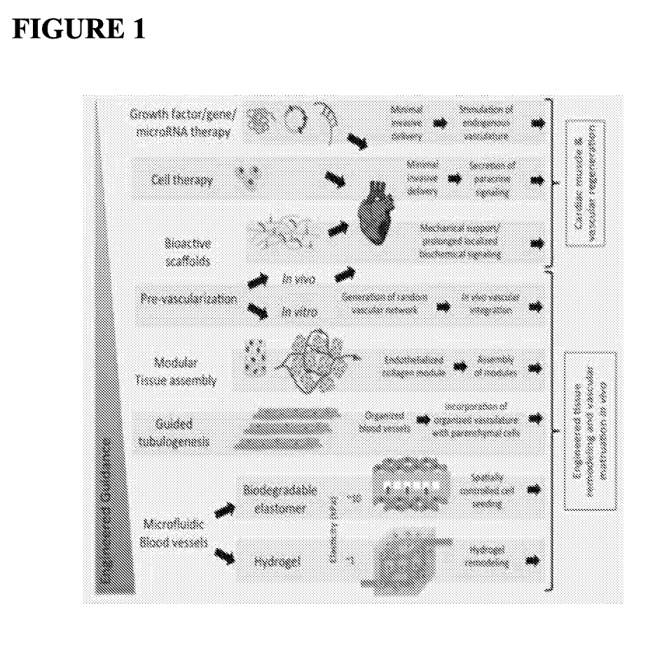

As modern micro-fabrication techniques mature and biomaterial science advances, direct-assembly of engineered blood vessels from single cells are beginning to show promise. Furthermore, the boundaries between biological and engineering strategies have begun to converge as recent approaches partially utilize micro-fabrication methods to help guide the natural assembly of blood vessels. Therapeutic cardiac vascularization strategies beginning from biological aspects of angiogenesis to recent engineered designs for growing microvasculature are highlighted with a focus on cardiac tissue engineering (FIG. 1).

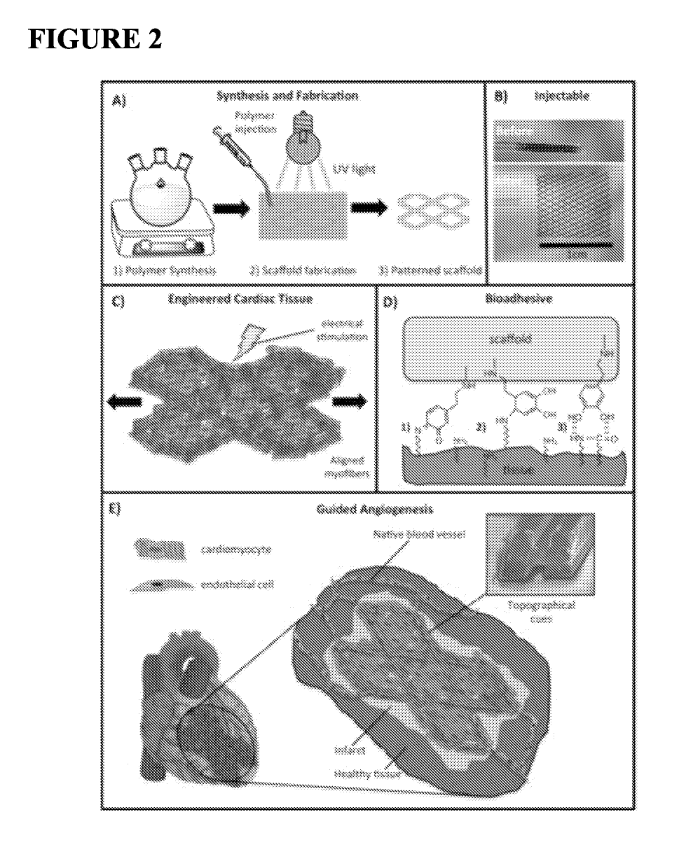

An overview of an exemplary system as described herein is depicted in FIG. 2. FIG. 2A illustrates the polycondensation and fabrication of a patterned scaffold. FIG. 2B shows an exemplary shape-memory scaffold before and after injection. In certain embodiments, wherein the tissue to be cultivated on the scaffold are cardiac myocytes, electrical stimulation can be applied in order to effectuate more complete and native-like cell differentiation and directional orientation of myofibers (FIG. 2C). In certain embodiments, the scaffold comprises a matrix including a dopamine-bioadhesive (FIG. 2D), which aids in attachment to the tissue. The dopamine moiety can be coupled to the polymer material of the scaffold fibers by, e.g., 1) Schiff base reaction with primary amine, 2) Michael addition with primary amine or 3) hydrogen bonding. FIG. 2E provides an illustration of topography guided blood vessel growth.

In the exemplary embodiment, several design parameters were initially outlined for the shape-memory patch: 1) Contain topographical cues to guide blood vessel sprouting; 2) Match anisotropic mechanical properties of the heart; 3) Biodegrade over an appropriate time-scale; 4) Posses shape-memory (regain its original shape) for minimally invasive delivery; and 5) Adhere to epicardial tissue (no suturing).

In the exemplary embodiment, a polymeric biomaterial was selected that is an elastomer because it has to endure thousands of stretch cycles without deformation or impeding heart contraction. The elastic biomaterial, called PoMaC (polyoctamethylene maleate [anhydride] citrate), is a photo-crosslinkable, biodegradable, nontoxic, and minimally inflammatory citric acid-based polymer. Pertinent design parameters such as how easy it is to work with (processability), replicating the stiffness of the myocardium (0.2-0.5 MPa) and ensuring that the material degradation rate matches the healing time scale of the heart, can all be fine-tuned by, e.g., controlling one or more of the monomer composition of the polymer, porogen content, degree of cross-linking. As such, a pre-polymer was synthesized through a polycondensation reaction. In an exemplary embodiment, a polymer was created at a molar ratio of 1 (citric acid):5 (1,8-octanediol):4 (maleic anhydride). The molar composition of acid to diol will be maintained at a 1:1 ratio while the feeding ratio of citric acid to maleic anhydride can be varied from (2:8 to 6:4).

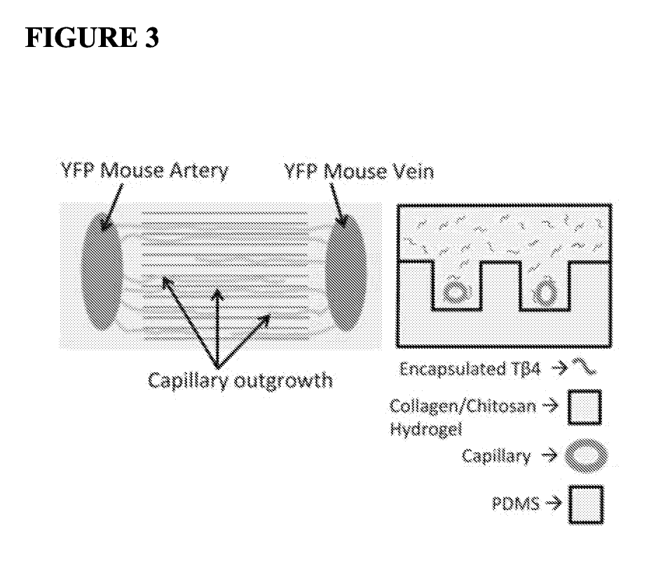

We reported in PNAS a new approach for creating an organized network of capillaries in vitro generated from an artery and vein using a micropatterned surface and a hydrogel that controllably released the peptide thymosin Beta-4 (Tβ4), which stimulated angiogenesis. An illustration of the experimental set-up can be seen in FIG. 3. In vitro data and mathematical modeling indicated that micropatterned surface geometries, called topographical cues, can enhance the sprouting of blood vessels by increasing local growth factor concentrations and also guide cell migration and elongation. As such, in certain exemplary embodiments, a channel is created in the scaffold fiber. The channel can be of any desired width; however, a channel width of about 50 μm produced the highest capillary outgrowth. Stimulating blood vessels to grow into the injured heart tissue would prevent the dead zone from expanding.

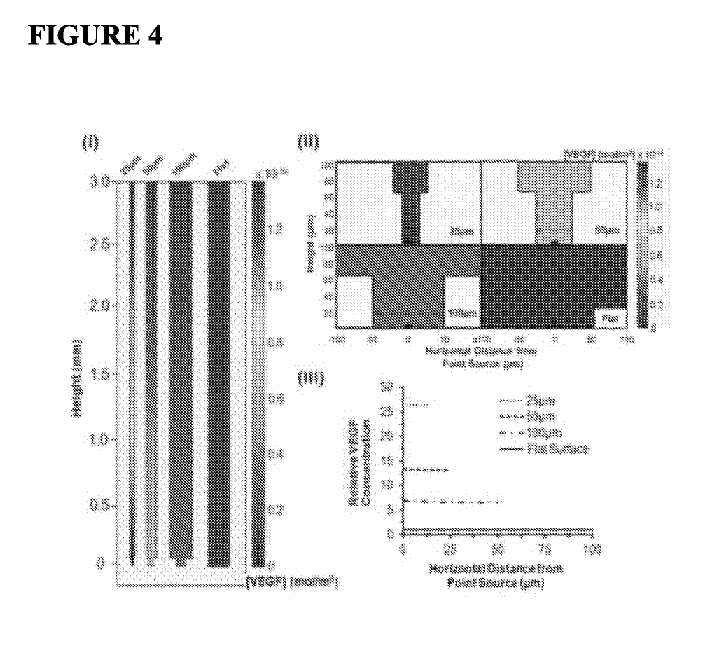

The concentration profiles of VEGF horizontally along the bottom of the microchannel for each diameter channel are shown in FIG. 4. In addition to providing physical topographical cues for the sprouting capillaries, the microchannels may also influence the local concentration of autocrine GFs released from the endothelial cells actively participating in angiogenesis. The effect that the physical barriers (provided by the microchannel walls) may have on these concentration profiles likely contribute to the differences observed in capillary growth vs. microchannel width. The model values are not absolute as several simplifying assumptions were used in its creation in order to observe the effect of microchannel geometry on local VEGF concentration.

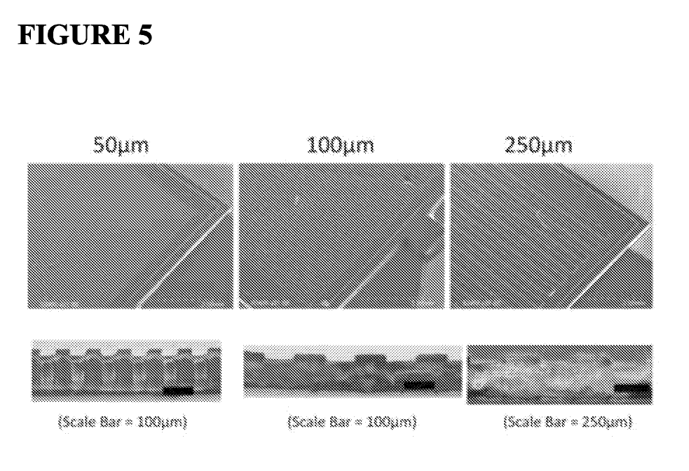

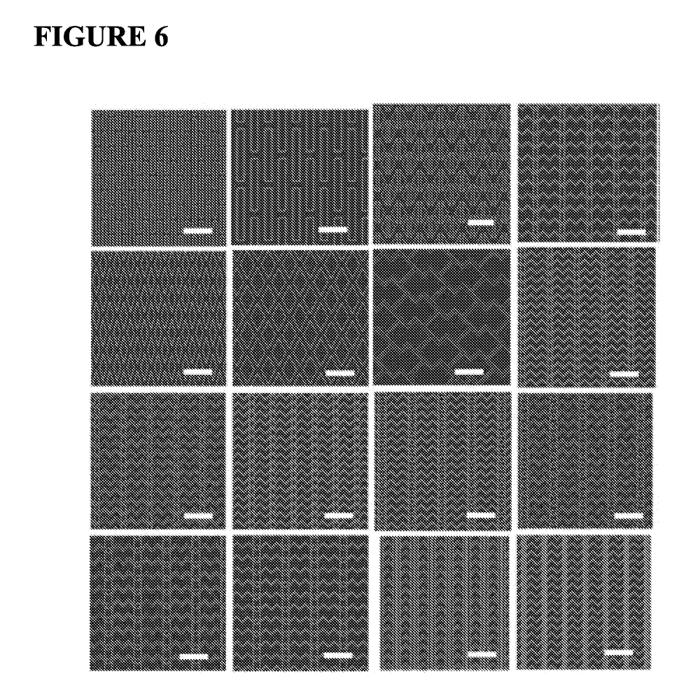

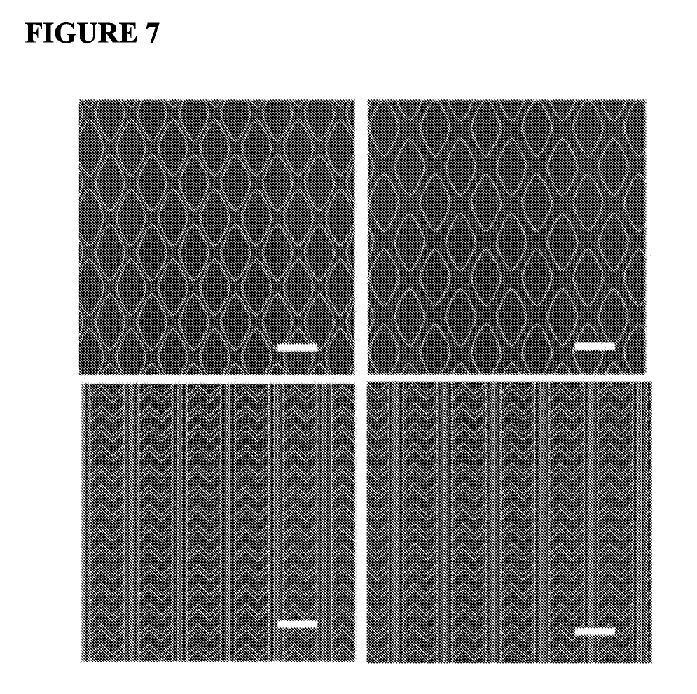

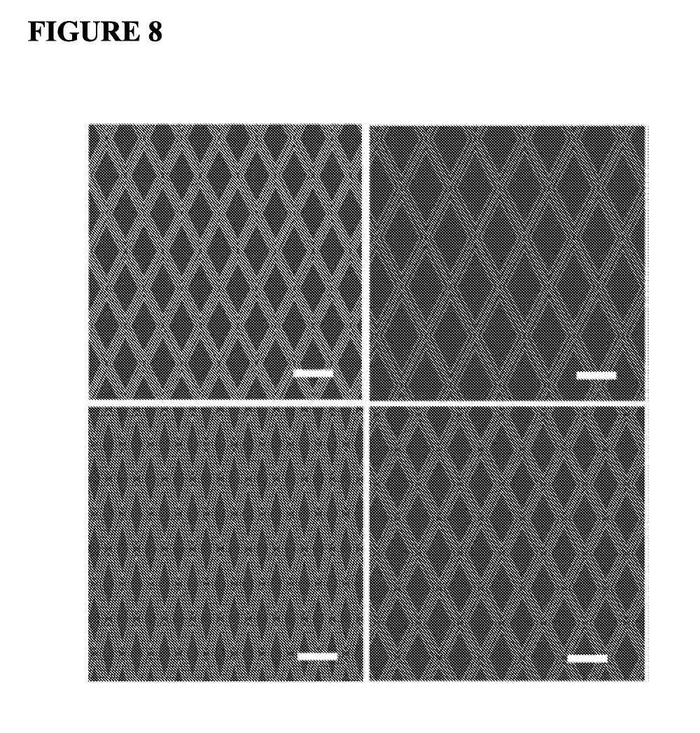

Microfabrication techniques were used to make a 1 cm² micro-patterned injection mold. The first generation of patterned scaffolds can be seen in FIG. 5. The mechanical properties of the scaffolds made of various polymer compositions and curing methods can be seen in Table 1. However, this design did not possess the desired anisotropic mechanical properties, injectability, or ability to not impede contraction of seeded cardiac myocytes (CMs). Subsequently, numerous scaffold designs that incorporated mechanical anisotropy and injectability were fabricated in iteration (See FIGS. 6-8).

TABLE 1
Mechanical data for scaffolds of various polymer compositions and
curing methods.

| | | | | Young's | | |
|---|---|---|---|---|---|---|
| | | | Curing | Modulus | Tensile | Elongation |
| CA | MA | OD | Method | (MPa) | Strength (MPa) | at Yield |
| 1 | 0 | 1 | Δ: 1 day | 0.47 ± 0.06 | 0.37 ± 0.06 | 2.34 ± 0.40 |
| 1 | 0 | 1 | Δ: 2 day | 0.61 ± 0.03 | 0.27 ± 0.12 | 1.03 ± 0.27 |
| 1 | 4 | 1 | Δ: 2 day | 0.16 ± 0.03 | 0.07 ± 0.03 | 0.97 ± 0.02 |
| 1 | 4 | 1 | UV | 0.27 ± 0.07 | 0.10 ± 0.03 | 0.39 ± 0.08 |
| 3 | 2 | 1 | Δ: 2 day | 0.99 ± 0.45 | 0.34 ± 0.10 | 0.52 ± 0.09 |

| | | | | |
|---|---|---|---|---|
| Rat | N/A | 0.001-0.14 | 0.03-0.07 | N/A |
| Myocardium | | | | |
| Human | N/A | 0.02-0.5 | 0.003-0.015 | N/A |
| Myocardium | | | | |

(Δ = 60° C.; CA: citric acid; MA: maleic anhydride; OD: 1,8-octanediol).

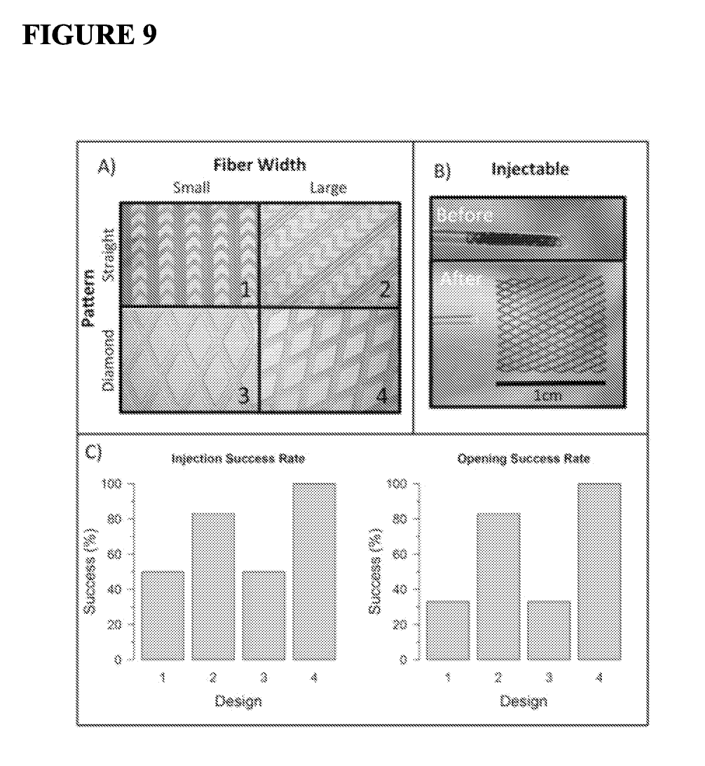

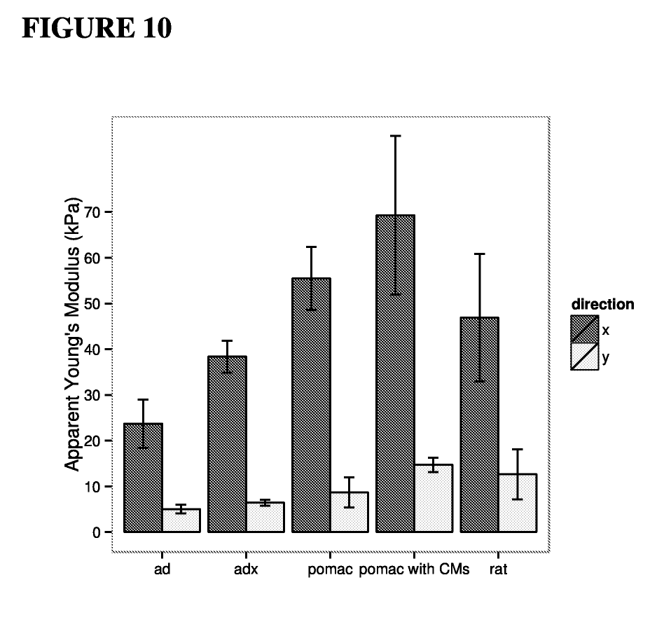

The shape-memory of the scaffolds were first assessed and eliminated if they did not regain shape or could not be injected. The final four designs that could be injected through the pipette are shown in FIG. 9A. After comparing the injection data it is apparent that the optimal design is Design 4 (FIG. 9B). The mechanical properties of this final design were then measured and summarized in FIG. 10.

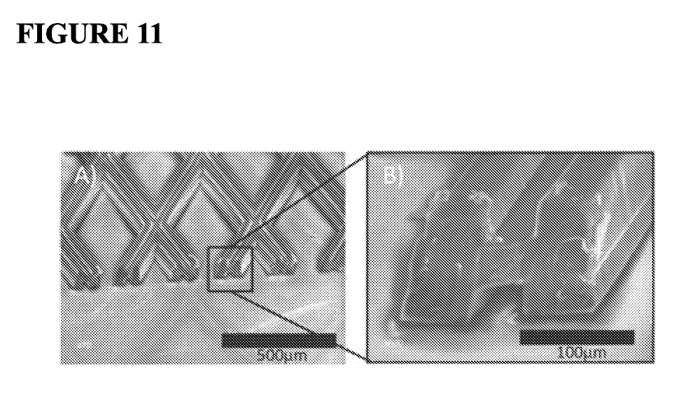

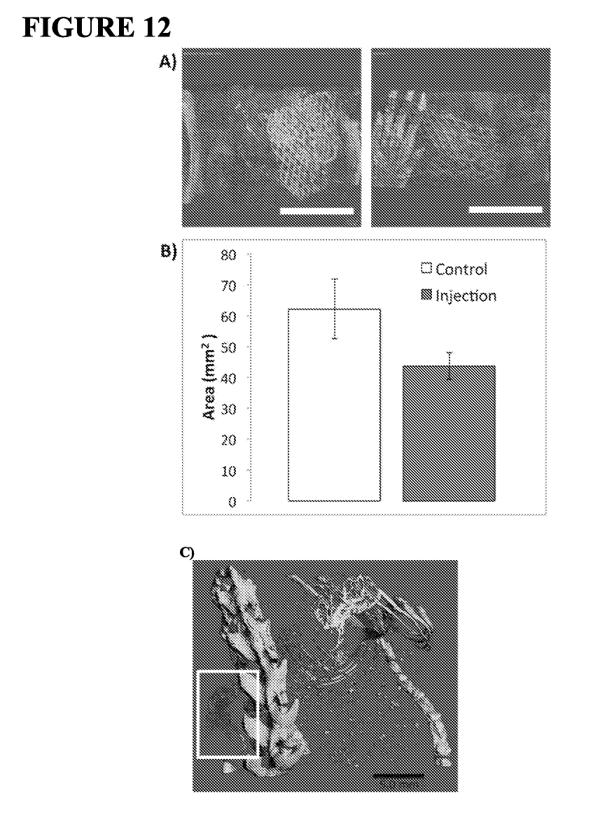

A double-channeled scaffold was also fabricated (FIG. 11) with the rationale that this would guarantee that the surface in contact with the tissue would have topographical cues on it. MicroCT is being used to characterize the scaffold shape-memory in vivo. An exemplary image can be seen in FIGS. 12A and C. FIG. 12B indicates that an exemplary injected scaffold as described herein re-opens after deployment approximately 70% of the area as compared to the control area.

Heart muscle is hierarchically organized ranging from macroscale bundles of aligned myofibers to the microscale repeating sarcomere units that permit cell contraction in response to electrical signals. Cardiac tissue engineering aims to replicate this structure by providing topographical and electrical cues to drive tissue maturation to resemble a functional adult-like state. Applying electrical stimulation to engineered cardiac tissue can drastically improve cell electrophysiological properties. Electrical stimulation was applied three days after neonatal rat cardiomyocytes (heart muscle cells) are seeded into the scaffold and cultured for two additional weeks to give optimal results. As the cells self-organize, internal tension is generated to promote aligned myofiber formation.

Confocal microscopy and immuno-fluorescence was used to characterize cell structure with specific cardiac proteins (sacromeric alpha-actinin, cardiac troponin T, connexin-43). Assays were also performed to assess for live/dead cells, and the excitation threshold (ET) and the maximum capture rate (MCR) were recorded before and after tissue injection to assess tissue robustness.

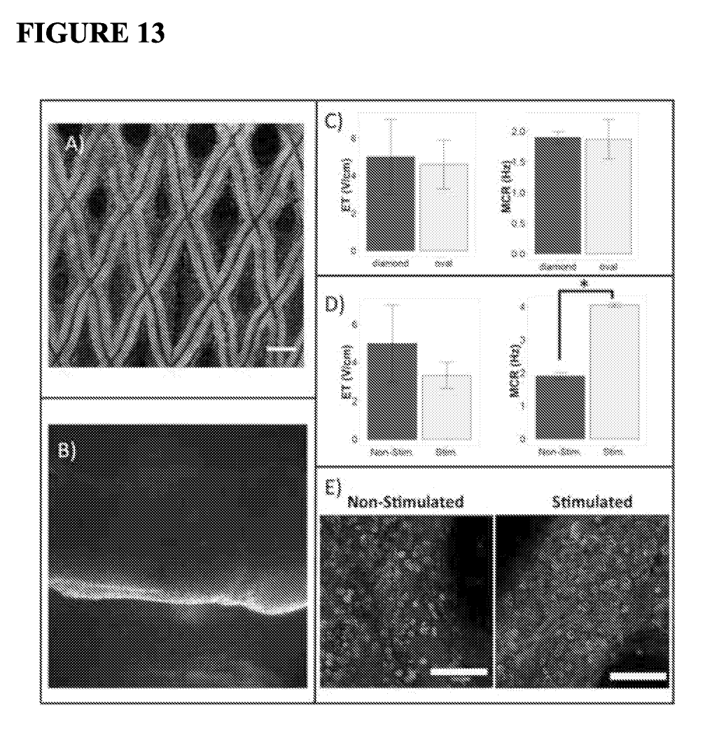

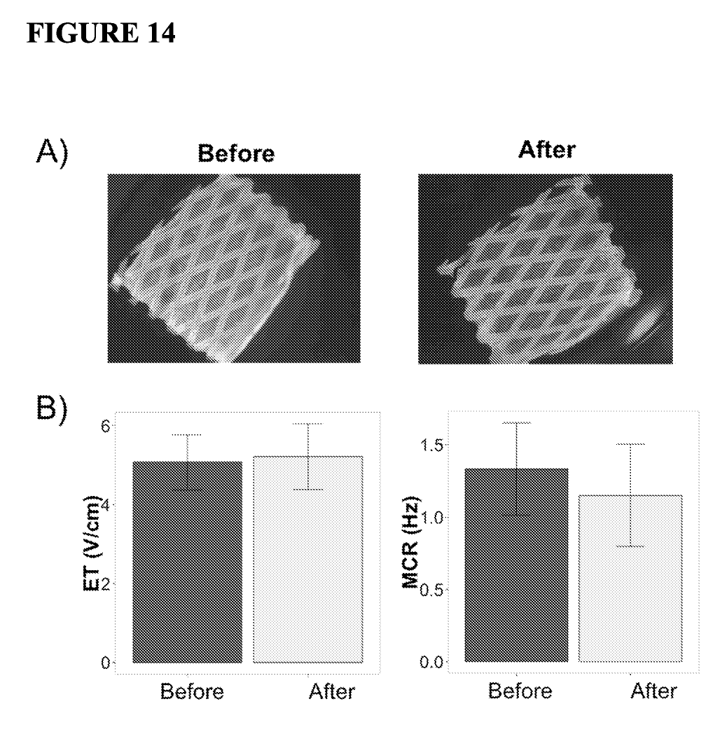

CMs were seeded and successfully cultured on the patterned scaffolds (FIG. 13A) and when injected subcutaneously into a rat the cells remained present (FIG. 13B). No statistical differences in the ET and MCR were observed between a diamond and oval patterned scaffold (FIG. 13C). Thus, the diamond pattern did not interfere with the cells remodeling the collagen Matrigel. When the cells were cultured under stimulation both the ET and MCR improved as expected (FIG. 13D). Comparing the structural staining between the nonstimulated and stimulated groups reveals improved alignment of the CMs and contractile proteins (FIG. 13E). The effect of injection on the cardiac sheets appears to have minimal damage on the seeded CMs (FIG. 14A). Further evidence that the injection process is non-damaging is provided in (FIG. 14B); there were no statistical differences in the patch electrophysiological properties.

However, the structural staining revealed that many of the CMs were rounded and not elongated. Without being bound by any particular theory, it is possible that many of these rounded CMs are not experiencing enough tension. Confocal imaging revealed that more cells are elongated in the diamonds that develop holes. As such, in certain embodiments, the shape-memory scaffold includes a void or hole in the area surrounded by the polymeric fibers.

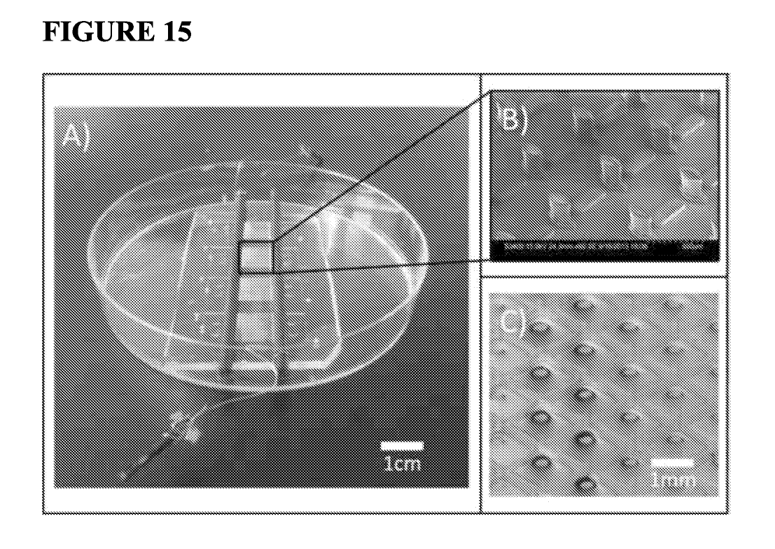

A bioreactor holder for the scaffold was fabricated (FIG. 15) so that more tension can be generated. Previously cardiac sheets were not secured during culture and over time developed curvature as the tissue matured. Furthermore, by including posts that protrude through the center of each diamond, holes will be created which will increase the internal tension to improve cell alignment. This will also make cell seeding more reproducible because initial cell seeding strategies resulted in scaffold detaching from the bottom surface and cells would slough off the scaffold. The purpose of the smaller posts is to lift the scaffold from the bottom of the PDMS so that when CMs are seeded they can go underneath the scaffold and surround the scaffold. In addition, the cell concentration can be reduced to, e.g., 250,000 CMs/mL so that more internal tension from gel compaction can occur which should improve cell alignment.

Thus, in certain embodiments, the shape-memory tissue scaffolds as described herein demonstrate one or more of the following features/advantages, including topographical cues to guide blood vessel sprouting; anisotropic mechanical properties of the tissue, e.g., heart; biodegrades over an appropriate time-scale; possess shape-memory (regain its original shape) for minimally invasive delivery (e.g., injectable delivery); adhere to tissue, e.g., epicardial tissue (no suturing); functional, e.g., functional engineered cardiac tissue has been cultured on the scaffold.

While not limiting in any way on the scope of the current disclosure, cardiac tissue was used as a model system because cardiomyocytes are extremely sensitive cells, immediate functionality of the heart tissue is desired, and this functionality can be easily assessed in vitro through measurements of contractile properties. The scaffold and methodology could be used with various cell types making this a versatile platform technology.

Bioadhesive Tissue Scaffolds.

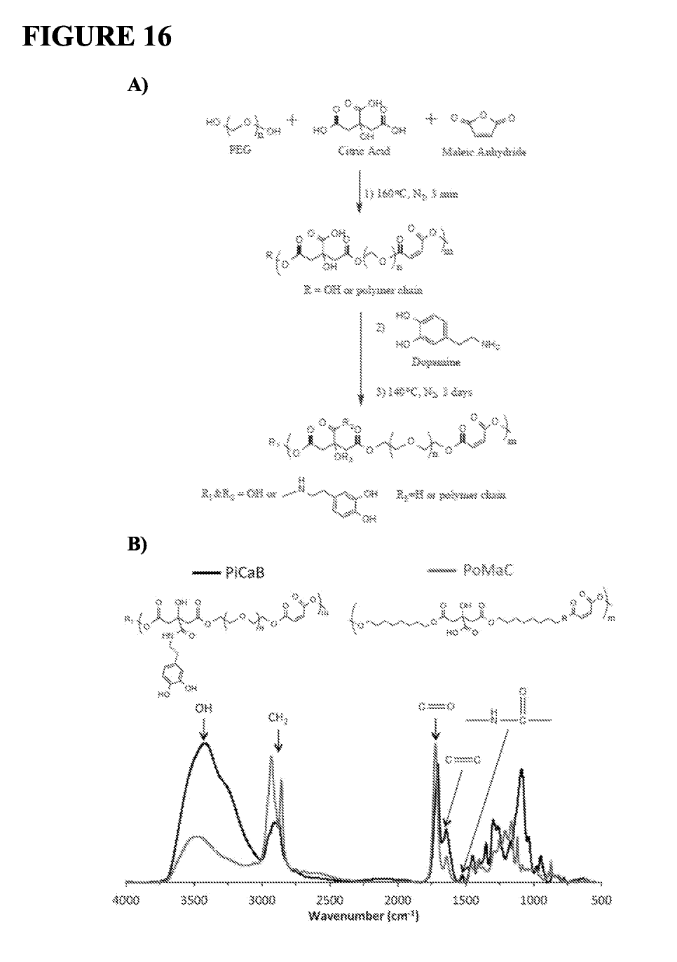

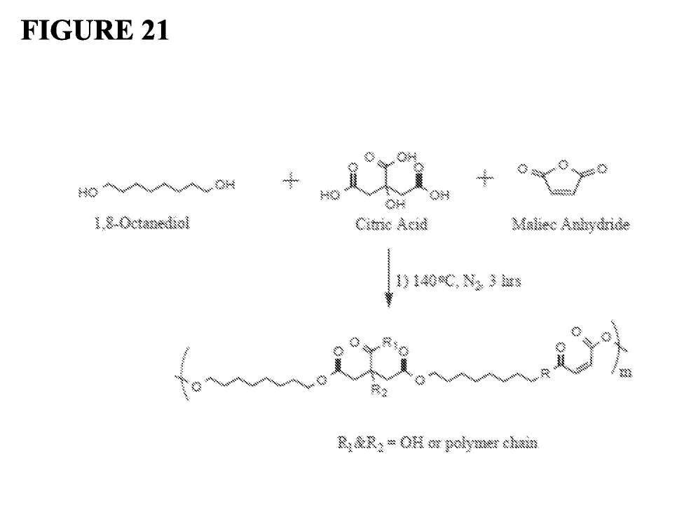

After an engineered tissue scaffold as described herein is prepared and the desired tissue is cultivated on the scaffold, e.g., cardiac tissue, it must be delivered to the treatment site, e.g., heart, and secured in place without sutures. This in vitro design criterion will be incorporated into the scaffold. A bioadhesive material as disclosed herein can be used for this purpose. Bioadhesives have been developed using a mechanism inspired by the way Zebra Mussels attach onto surfaces with dopamine. In one aspect, the disclosure provides an acid-based polymer fiber, e.g., citric acid-based polymer fiber that not only contains dopamine but a photocrosslinkable double bond from maleic anhydride. This advanced biomaterial maintains the desirable aforementioned properties of PoMaC. The reaction scheme for the bioadhesive can be seen in FIG. 16. Scaffolds were made out of an elastic, biodegradable, dual cross-linkable (heat and UV) thermosetting polymer. The two-step polycondensation reaction scheme for producing a biodegradable dual cross-linkable bioadhesive polymer is shown in FIGS. 16A and 21. In certain embodiments, citric acid is replaced with 1, 2, 4-butanetricarboxylic acid. FIG. 16B shows ATR_FTIR spectra of PiCaB and PoMaC pre-polymers.

Briefly, polycondensation of 1,8-octanediol, maleic anhydride, and citric acid (Sigma-Aldrich) were added to a 250 mL triple-neck flask in a molar ratio of about 5:1:4, respectively. The moles of carboxylic acid groups to moles of hydroxyl groups present remained equimolar. The reaction vessel was heated to about 160° C. and stirred shortly until a clear solution was formed before subsequently decreasing the temperature to about 140° C. for about 3 hours under nitrogen purge. The poly(octamethylene maleate (anhydride) citrate) (POMaC) pre-polymer was then dissolved in 1,4-dioxane and purified by drop-precipitation into deionized water followed by about 3 days of lyophilization. The purified POMaC pre-polymer solution was then mixed with the porogen, poly(ethylene glycol) dimethyl ether (PEGDM, Mw˜500, Sigma) at 60 wt % and 5 wt % UV initiator 2-hydroxy-1-[4(hydroxyethoxy)phenyl]-2-methyl-1 propanone (Irgacure 2959). The final mixture was stored in the dark at room temperature.

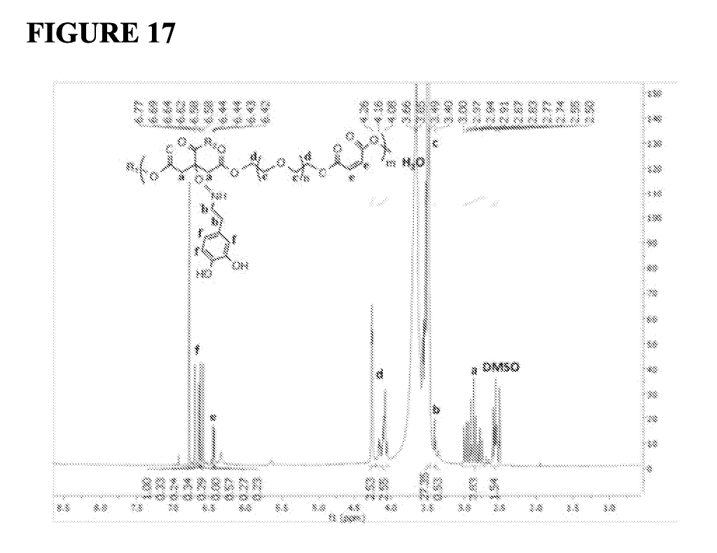

With reference to FIG. 17, which shows the ¹H-NMR spectra for the dopamine bioadhesive polymer (PICAB), the chemical shift (δ) at 2.5 is due to the solvent DMSO. The δ's from 2.74-3 (a) are due to the H's in the backbone of the citric acid 153. The large peak at a (δ) of 3.5 (c) confirms the presence of protons from PEG and the shifts between 4.08 to 4.26 (d) were assigned to methylene groups of PEG adjacent to an ester bond 154. The peak at 3.65 was assigned to water because it was associated with PEG, normally H₂O in DMSO has a δ of 3.30. The δ at 6.44 (e) represents that protons adjacent to alkene group in the z-position 140. There also appears to be a large amount of water present associated with PEG by the increased δ at 3.65. A triplet at 6.62, 6.64, and 6.69 (f) were assigned to the three aromatic hydrogen molecules from dopamine 155. They have a slightly lower δ compared δ's commonly seen in aromatic hydrogen due to the oxygen's of the hydroxyl groups donating electrons. They also all have an equal integral value of ˜0.3 indicating the correct ratio. The hydrogen's in the alkane in dopamine are present due to the triple δ at 3.40 (b). The integral of the triplet is ˜0.6, which is as expected double the integral of the aromatic hydrogen's which further confirms the presence of dopamine.

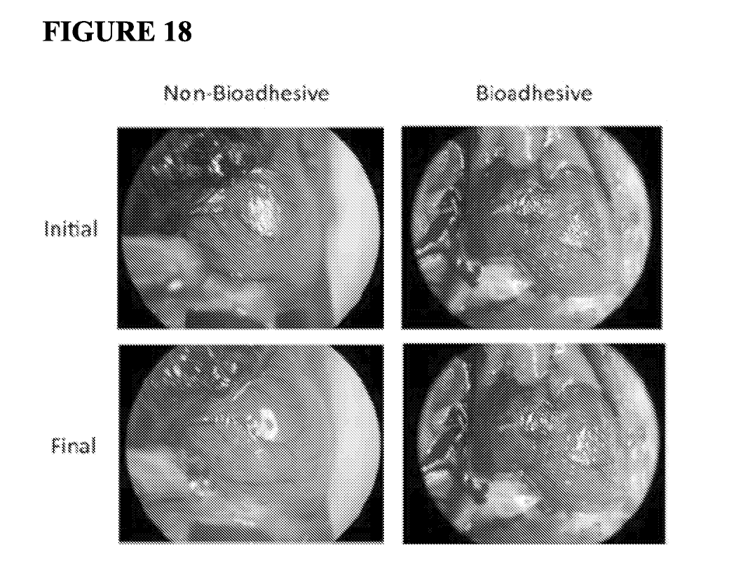

A non-adhesive and adhesive scaffold were each placed onto a rat heart and rinsed vigorously with PBS to try and detach the scaffolds from the tissue (FIG. 18). The scaffold made from PICAB, when oxidized, did not detach from the surface but PoMac did.

A simplified mathematical model was formulated in order quantify potential differences in local autocrine GF concentrations due to different microchannel widths. A novel microfabrication strategy was used to fabricate various patterned PoMaC scaffolds. An optimal scaffold design with shape-memory and topography has been identified through a series of in vitro injections. The anisotropic mechanical properties of the scaffold closely matched the native rat myocardium. Functional cardiac sheets with shape-memory were created and successfully injected through a 1 mm orifice. A novel dual cross-linkable (light and dopamine) biodegradable bioadhesive polymer was synthesized through a polycondensation reaction. The pioneering developments of new shape-memory biomaterial scaffolds have a potential to revolutionize the field of tissue engineering by enabling minimally invasive delivery of functional tissue.

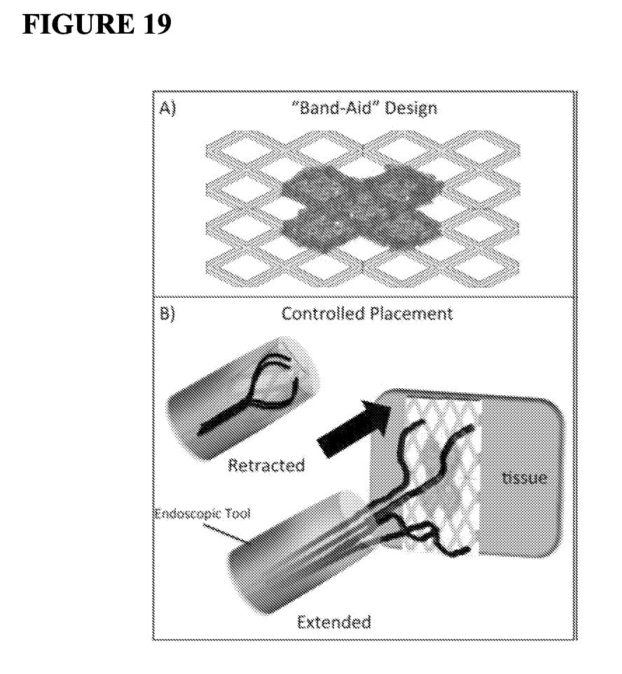

In an additional aspect, the disclosure provides a tissue scaffold “band-aid.” In certain embodiments, this design may have CMs on the scaffold with a perimeter of cell-free scaffold. The purpose is to provide a region where the bioadhesive could attach onto the surface of the tissue, e.g., heart (See FIG. 19).

A potential issue with the direct injection method is that it could be difficult to determine where the patch will end up after it has been ejected. Thus, in still an additional aspect, the disclosure provides a direct injection device and method. In an embodiment, the device comprises an endoscope having a lumen and reversibly retractable tines, wherein the tines can be retractable into the lumen in order to fold a shape-memory tissue scaffold within the lumen for insertion into a subject, and wherein the tines can be extended to deploy and reopen the shape-memory tissue scaffold when apposed or near the desired tissue site. An exemplary embodiment is depicted in FIG. 19.

A representative 2D steady-state model of VEGF₁₆₅ diffusing out of a single cell was solved using finite element model software (Comsol 3.5). The solver used was DIRECT(UMFPACK). All initial concentrations were equal to 0. As this model was to simply investigate the effect of geometry on a local VEGF concentration, several complexities (e.g. kinetic receptor and ECM binding, basement membrane formation, proteolysis, internalization, multiple VEGF splice isoforms, etc.) were ignored.

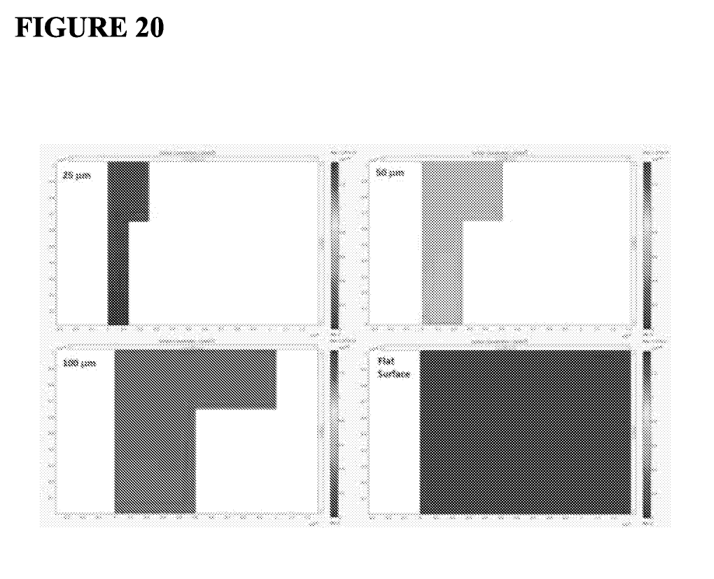

In the design of the microchannel cross-sections (FIG. 20), advantage was taken of the inherent symmetry of the repeating microchannels. This allowed us to draw one-half cross-section (to scale) of an arbitrary individual microchannel on a 2D (x-y) grid. The total height of the model was the actual height of the culture medium above the surface, which was calculated from the volume of the culture medium, dimensions of the PDMS stamp, and the area of the cell culture well. A quarter of a 10 μm diameter circle (centered at 0,0) was used to emulate a cell releasing VEGF from the bottom of a channel. The measured thickness of the collagen gel was incorporated into the model, as the diffusivity of VEGF will be hindered in the collagen-based gel. The case of a flat surface was also modeled in the same manner but the total width was equal to the radius of a 12 well cell-culture plate.

The aqueous diffusivity of VEGF₁₆₅ (D_{165, H2O}) at 37° C. used (1.30×10⁻¹⁰ m²/s) was calculated using the Stokes-Einstein relation and data from Berk. The steady-state diffusion-reaction for each subdomain was defined by the following equation:

∇·(−D₁₆₅·∇o₁₆₅)=R₁₈₅

where D₁₆₅ is either D_{165, H2O} or the effective diffusivity of VEGF₁₆₅ in collagen (m2/s), c₁₆₅ is the VEGF₁₆₅ concentration (mol/m³), and R₁₆₅ is the production rate of VEGF₁₆₅ (mol/(m³·s)) from the cell.

The rate of VEGF production (0.048 molecules/cell·s) was assumed constant 135. The boundary conditions for the left and right borders were treated as insulation/symmetry boundaries as there will be no flux of material through these symmetry lines. The boundaries representing PDMS were considered to be impermeable to proteins (i.e. VEGF) and therefore were treated as insulation boundaries. To validate this assumption the diffusivity of BSA (DBSA) in water or PDMS were compared. The DBSA in water is about 5.97±0.44×10⁻¹¹ m²/s while the diffusivity of BSA in PDMS was reported to be 2.69±0.1×10⁻¹³ m²/s which is two orders of magnitude smaller.

Furthermore, PDMS has been shown to have a molecular weight cut-off (MWCO) of ˜1000 g/mol and has been used in organic solvent nanofiltration systems. Therefore, as the MW of VEGF₁₆₅ is much larger than this (˜39 kDa), an impermeable PDMS boundary can be assumed. The boundary condition for the top layer was set to a concentration of zero.

Interlocking Tissue Scaffolds.

Complex hierarchical cellular alignment is omnipresent in the human body, such as in blood vessels, neural networks, and cardiac or skeletal muscle. These structural features translate into critical functional characteristics. For instance, the highly organized and integrated pseudo-laminar myocardial syncytium correctly distributes an electrical propagation front that translates into orchestrated cardiac fiber contraction. The myocardium is also comprised of multiple cells types. The co-culture of multiple cell types has well-known to improve the functionality and survival of cardiac tissue in vitro and in vivo. Furthermore, the native myocardium contains sheets of fibroblast layers. Therefore, the ability to control the co-culture arrangement of engineered tissue constructs is a desirable feature. Traditional tissue culture methods such as embedding cells on foam scaffolds with a random pore distribution or a uniform hydrogel have been implemented to cast thick tissues rapidly, but often lack control over the intercellular organization required for organized tissue assembly.

To mimic cellular and tissue level organization, tissue fibers have been engineered using microfluidic devices by extruding cell-embedded fibers comprised of calcium alginate (Ca-alginate) or chemically modified gelatin. This strategy miniaturized the engineered scaffold (hydrogel fibers) to provide topographical guidance in the micro-scale achieved guided cell assembly in 1D. However, the assembly of these engineered tissue-fibers into 3D tissue is tedious, requiring bundling, reeling, and weaving. Thin accordion-like honeycomb mesh or rectangular mesh (100-200 μm thick) micro-fabricated scaffolds made of a biodegradable elastomer has also been used to culture cardiomyocytes (CMs) with induced topographical cellular alignment. The patterned scaffold mesh provided an anisotropic stiffness that mimicked the native myocardium. This strategy creates cellular organization in the 2-D, but the assembly of multiple scaffold meshes into 3-D tissue while preserving the organized tissue structure has not been demonstrated.

To accelerate this tissue assembly process, an interlocking tissue scaffold system is provided. The system is a micro-fabricated, biodegradable scaffold mesh that provides structural cues to instruct cellular alignment into organized fiber mesh in 2D while allowing rapid 3D tissue assembly through a hook and loop mechanism similar to conventional Velcro®. To demonstrate the feasibility of our approach, we used CMs to construct a functional cardiac tissue with aligned fibers in 3D.

Natural extracellular matrices (ECMs), such as collagen and matrigel, were used to facilitate matrix remodeling into an engineered cardiac tissue that reconstitutes native cellular morphology and function. A synthetic elastic biodegradable polymer core scaffold provides mechanical stability and allows manual handling and assembly. The scaffold mesh design also provides anisotropic mechanical stiffness designed to mimic the native myocardium. The interlocking tissue scaffold design provides a topographical feature that allows multiple cell types to be cultured individually and then assembled together vertically or horizontally to establish a co-culture system.

Scaffold Fabrication.

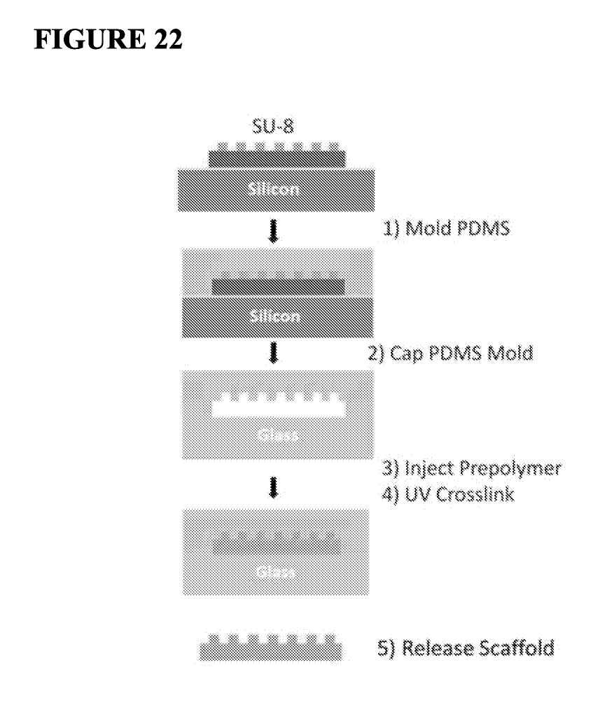

Scaffolds were fabricated using standard SU-8 photolithography techniques as previously described and an illustration of the overall procedure can be seen in FIG. 22. Briefly, SU-8 2050 photoresist (Microchem) was spin-coated on silicon wafers according to manufacturer guidelines. SU-8 photoresist was exposed to 365 nm, 11 mW/cm² UV light using a mask aligner (Q2001, Quintel Co., San Jose, Calif.) through transparent masks. The multi-layered device required proper alignment between the features on the first and second layers before exposure. Finally, the master mold was submersed in SU-8 developer solution until all the unexposed photoresist was dissolved from the surface. A negative of the mold was made by pouring poly(dimethylsiloxane) (PDMS) elastomer with a curing agent (20:1 ratio) and curing at room temperature for about 3 days. Holes were punched into the inlet and outlet of the PDMS molds with a 21G borer. The PDMS molds were then capped with a glass slide by static adhesion to form a closed network of channels. The POMaC pre-polymer/porogen/UV initiator mixture was then slowly injected through mold at the inlet and left overnight before UV exposure to allow trapped air bubbles to dissipate. The PDMS molds were exposed at 16 mW/cm2 for 3 min followed by peeling PDMS molds from the glass caps. The partially cross-linked POMaC solution adheres strongly to the glass due to hydrogen bonding and remains attached to the glass slide. The cross-linked polymer scaffold was then removed from the glass substrates and placed in PBS to leach out the PEGDM creating a nanoporous scaffold. Multi-layered molds can be made (e.g. a scaffold with features both on the top and bottom or stacking layers) using either a molded or flat PDMS cap instead of the glass slide.

However, when peeling off the PDMS cap the work of adhesion is higher in the PDMS mold because of the larger surface area from the features. This means that the cross-linked POMaC remains in the PDMS mold. Next, a PDMS mold and glass slide with the scaffold can be aligned and pressed together and exposed to covalently bond the two layers together.

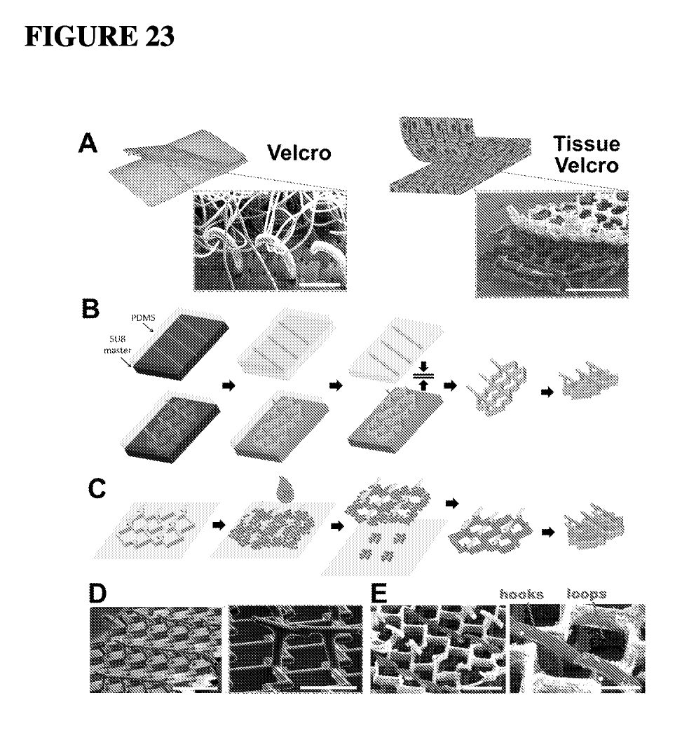

We envisioned designing living tissues that could be as easily and firmly assembled as two pieces of Velcro®. Conventional Velcro® (FIG. 23A) is composed of two sheets: one sheet is an array of hooks and the other is a sheet of fibers that form loops. When the two surfaces are brought into contact the loops catch on the hooks and the layers remain attached until a sufficient pull-off force is applied. Not all hooks will attach to a loop, but when a sufficient number of hooks “catch” a loop over the contact area, significant adhesive force can be generated. The interlocking tissue scaffold system as described herein uses the same mechanical interlocking principle to lock two living tissue mesh together (FIG. 23B).

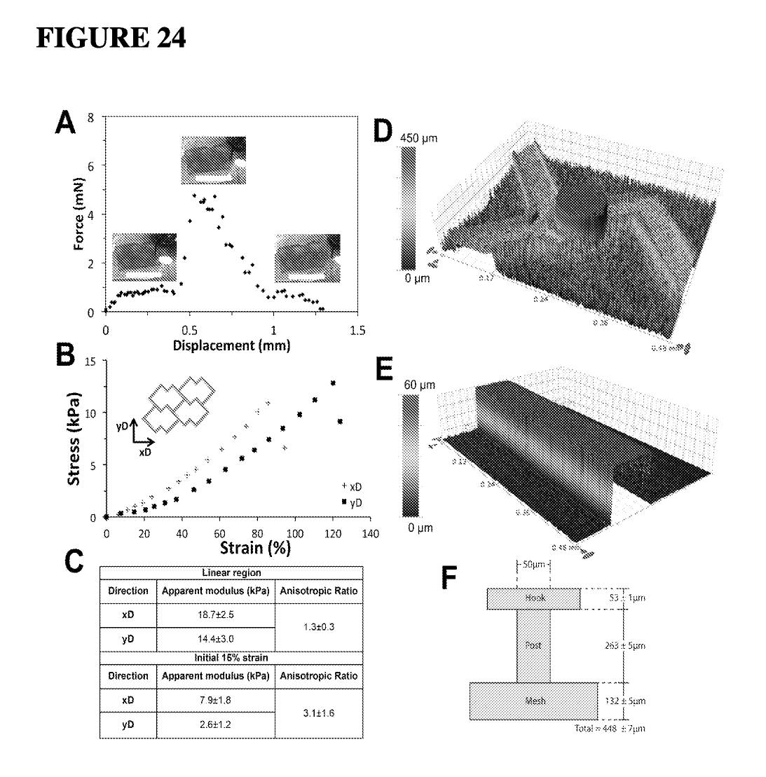

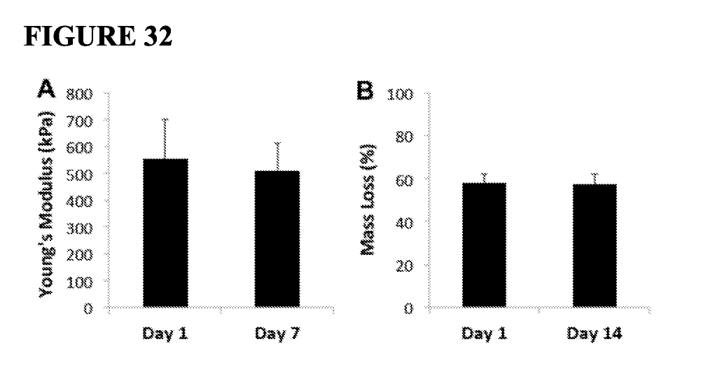

First, an accordion honeycomb mesh was fabricated via injection molding of a biodegradable elastomer, poly(octamethylene maleate (anhydride) citrate) (POMaC) (FIG. 23B). POMaC is a biodegradable and UV-photocrosslinkable elastomer prepared through polycondensation reaction from the monomers (1,8 octandiol, citric acid, and maleic anhydride) under mild conditions 17. The bulk material exhibited a negligible drop in Young's modulus from day 1 to day 7 in culture media in the presence of cells (FIG. 32A). The bulk material mass loss was also negligible from day 1 to day 14 (FIG. 32B), while the initial mass loss could largely be attributed to porogen leaching during the rinsing step. The void spaces within the accordion honeycomb mesh function as the loops of the conventional Velcro system. Small T-shaped hooks were patterned by aligning and bonding a horizontal rectangular cap onto the posts on the base mesh (FIG. 23B). The cap was transferred with a PDMS substrate, and the bonding was achieved by UV cross-linking (FIG. 23B). In the last fabrication step, the cap was incised to break the connection between the posts, establishing individually standing T-shaped hooks (FIG. 23B). In the exemplary embodiment, the T-shaped micro-hooks have struts that are about 50 μm wide and about 250 μm tall. The height of the micro-hooks is sufficient to protrude through the void space of another scaffold mesh and anchor onto its struts. The scanning electron microscope image of two tissue meshes brought into contact shows the locking mechanism where the hooks of the bottom scaffold protrude through the mesh of the top scaffold and “lock” the two meshes together (FIG. 23D). The maximum force recorded to pull-off the scaffold was about 6.21±1.12 mN, or when divided by the area of the scaffolds (2.5×5 mm), the pressure required is about 0.50±0.09 kPa. A representative plot of the pull-off test is shown in FIG. 24A. The binding force between the two meshes is sufficiently strong to withstand manual manipulation such as stretching or compression. The accordion honeycomb pattern was chosen so that the scaffold exhibited spring-like elasticity as well as anisotropic stiffness in the x-y plane (FIG. 24B). The scaffold mesh displays anisotropic mechanical properties with an anisotropic ratio of about 1.6±0.18 with the apparent modulus greater in the x-direction compared to the y-direction.

Although the exemplary scaffold mesh was made in an accordion honeycomb pattern, the invention is not so limited. As would be understood by the skilled artisan, the mesh can be of any desired geometric configuration or a random mesh (e.g., electrospun fibers) so long as voids or spaces are present that allow for the interlocking of micro-hooks. In addition, the interlocking tissue scaffold system can be combined with the shape-memory tissue scaffold system as described herein.

Cell seeding was achieved by pipetting a cell suspension in Matrigel onto the scaffolds, allowing partial gelation (FIG. 23C), and then the scaffold was immediately lifted off the plastic tissue culture polystyrene substrate, allowing only the cells close to the scaffold struts to remain attached, thus producing small holes in the tissue (FIG. 23C). Culture was continued to allow for cell self-assembly (FIG. 23C). Subsequently, tissue patterning or stacking was performed with multiple tissues (FIG. 23C).

The micro-hooks of a single layer scaffold, which protrude through the void space of another scaffold mesh and anchor onto its struts, were imaged with a scanning electron microscope (SEM) (FIG. 23D). The SEM image of two tissues brought into contact shows the attachment mechanism where the hooks of the bottom scaffold protrude through the honeycomb mesh of the top scaffold and affix the two tissue meshes together (FIG. 23E). The maximum force recorded to pull-off the scaffold was 6.2±1.1 mN, or when divided by the area of the scaffold (2.5×5 mm), the pressure required was 0.5±0.1 kPa. Typically 18 hooks (equivalent to 82% of total hooks) will successfully lock in place across the scaffolds when two 2.5×5 mm layers are brought into direct contact without offset. A 3D reconstruction from a confocal z-stack of an assembled 2-layer scaffold construct shows the interlocking mechanism.

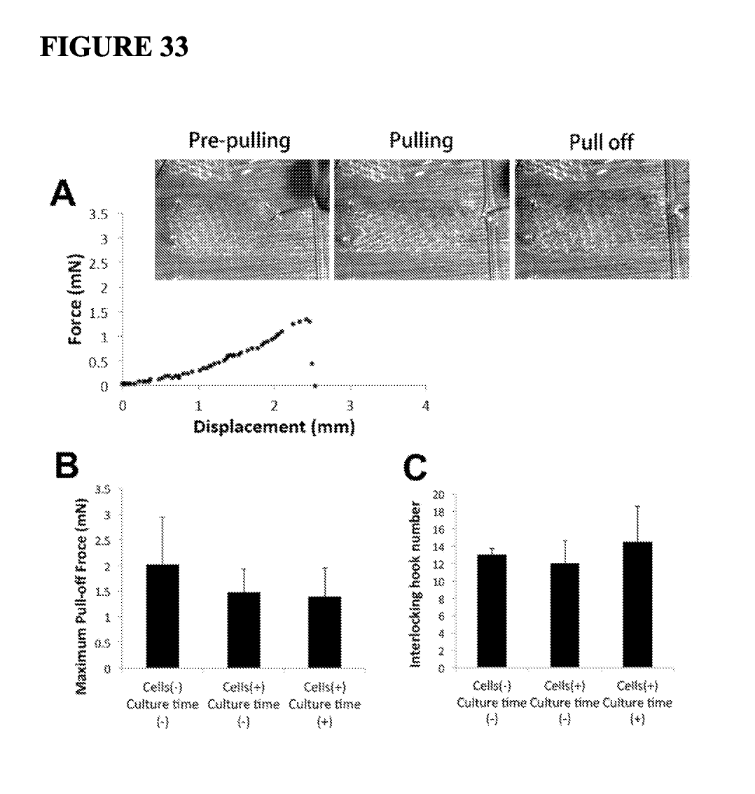

A representative plot of the pull-off test is shown in FIG. 24A. The binding force between the two scaffolds is sufficiently strong to withstand manual manipulation such as stretching or compression. The presence of cells on the scaffold or a short culture time between two layers (3 days) did not significantly affect the pull-off force (FIG. 33). Thus, the hook and loop interlocking mechanism was primarily responsible for the mechanical stability of the assembled layers. The pull-off force was significantly higher when two scaffolds were overlaid by 100% (FIG. 24A, 6.2±1.1 mN) in comparison to measurements in partially overlaid scaffolds (FIG. 33B, 2.0±0.9 mN, p=0.001) as expected.

The accordion honeycomb pattern was chosen so that the scaffold exhibited spring-like elasticity, topographical cues for cell alignment, and anisotropic stiffness in the x-y plane as described by Engelmayr et a18 (FIG. 24B, C). In the linear region of the curve, the scaffold mesh displayed anisotropic mechanical properties with an anisotropy ratio of 1.3±0.3. The apparent scaffold modulus was greater in the long axis (xD) direction (18.7±2.5 kPa) compared to the short axis (yD) direction (14.4±3.0 kPa, n=4, p=0.067). However, the scaffold strain expected from cell contraction is lower than the strain exhibited within the linear region (FIG. 2B). Within the physiological regime of scaffold strain of up to 15% as described 8, the scaffold mesh displayed anisotropic mechanical properties with an anisotropy ratio of 3.1±1.6 and the apparent modulus significantly greater in the xD (7.9±1.8 kPa) compared to the y-direction (2.6±1.2 kPa, n=4, p=0.002). The feature heights of the scaffolds were measured using a prolifometer, resulting in 53±1 μm tall hooks, positioned on top of 263±5 μm tall posts protruding off of the 132±5 μm thick mesh base for a combined total height of 448±7 μm (FIG. 24D,F).

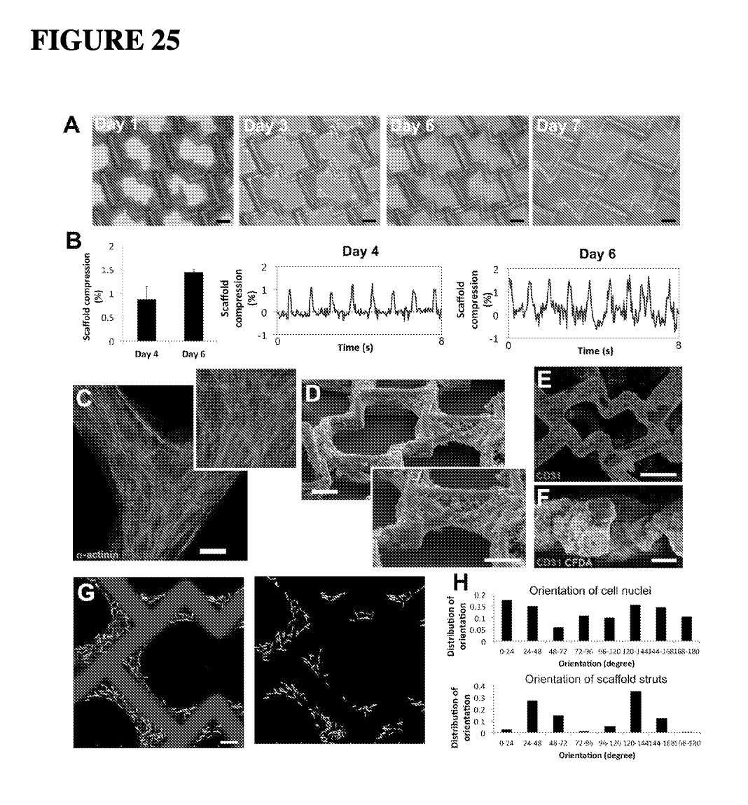

The fibers of the mesh provided topographical cues to guide cellular assembly in the x-y plane. Neonatal rat CMs were seeded onto the scaffolds with Matrigel, where the cells initially wrapped around the struts of the mesh and then remodeled the matrix by compacting and elongating around the struts over a period of 7 days (FIG. 25A). After 4-6 days, the tissues displayed spontaneous contraction. Cardiac tissue contraction was paced using an electrical stimulator. As the tissue contracted, it compressed the scaffold in a springlike fashion. Scaffold autofluorescence allowed for the deformation of the scaffold mesh under fluorescent microscopy to be tracked with image processing. The degree of scaffold compression was characterized by tracking the decrease in the honeycomb area during contraction. A trend toward higher scaffold compression (% area decrease at each beat) was recorded at day 6 compared to day 4 (Day 4: 0.87±0.27%, Day 6: 1.44±0.07%, n=3) (FIG. 25B).

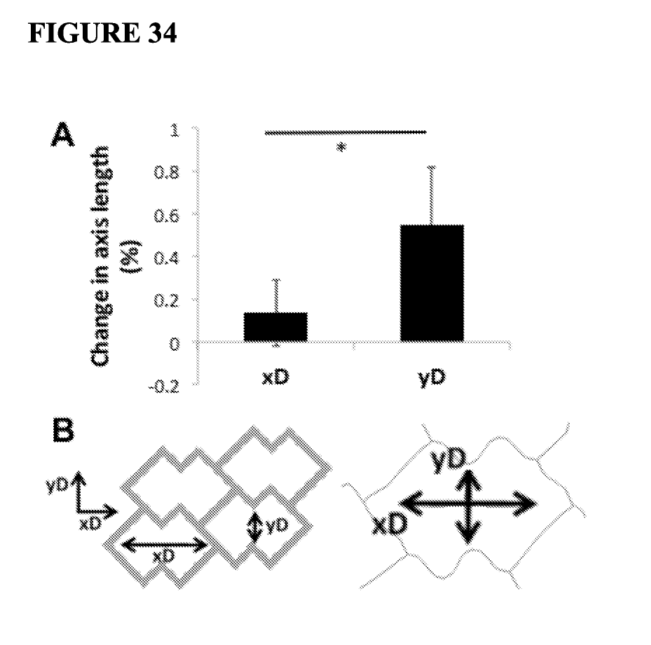

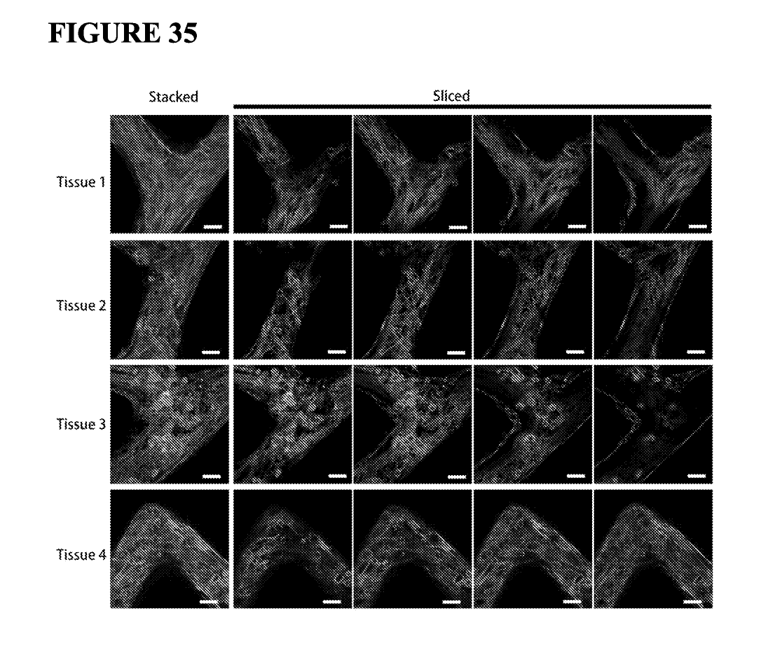

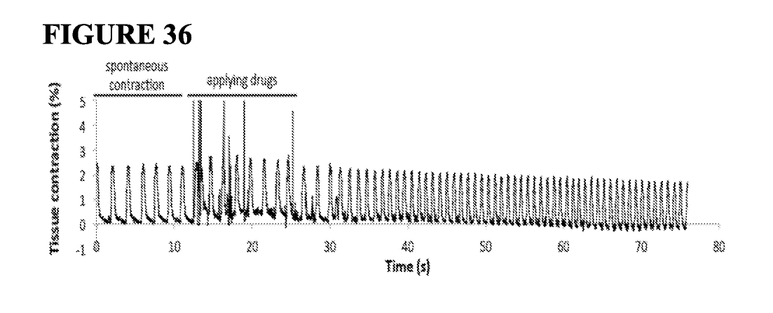

On day 8, the linear percent shortening was higher in the short axis (yD) direction than in the long axis (xD) direction (p=0.038) (FIG. 34), consistent with the lower modulus in the short axis direction allowing for greater deformability (FIG. 2B). Immuno-fluorescence staining of the cytoskeletal actin filament, F-actin, and contractile protein, sarcomeric α-acitinin, and SEM revealed formation of a tissue layer with elongated cardiomyocytes around the scaffold struts and visible cross-striations (FIG. 25C,D, FIG. 35). Cardiac tissue was also able to exhibit a positive chronotropic response upon exposure to 300 nM epinephrine (FIG. 36).

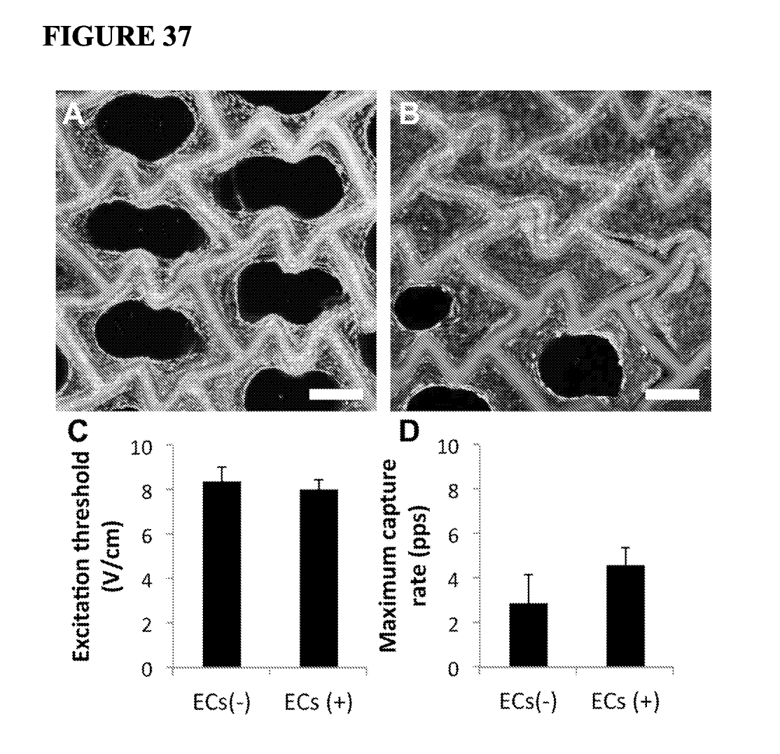

The compatibility of interlocking tissue scaffold with conventional co-culture techniques was demonstrated by coating an additional layer of endothelial cells (ECs) on heart cells compacted around the mesh. This was achieved by adding an EC suspension to the tissue mesh for 24 hr and cultivating in the EC culture media. CD31 immuno-fluorescent staining revealed a near confluent coating of ECs with cobblestone-like morphology around the tissue (FIG. 25E). A cross-sectional view of the tissue mesh coated with ECs co-stained with live cell tracker and CD31 confirmed the ECs covered the surface of the tissue and the heart cells occupied the inner core (FIG. 25F). The EC coating provided an additional dimension in the co-culture assembly, a beneficial feature if the entire tissue is to be perfused through its void spaces, where ECs can function as a barrier to shield the parenchymal cells from fluid shear stress. When culturing Interlocking tissue scaffold within an orbital flask bioreactor in EGM-2 media at 160 RPM with or without EC coating, EC coating helped to better maintain tissue structure (FIG. 37). Scaffold guidance of cellular alignment was confirmed by comparing the normalized distribution of cell orientation measured from the main axis vector of the nuclei to the distribution of scaffold strut orientation (FIG. 25G, H).

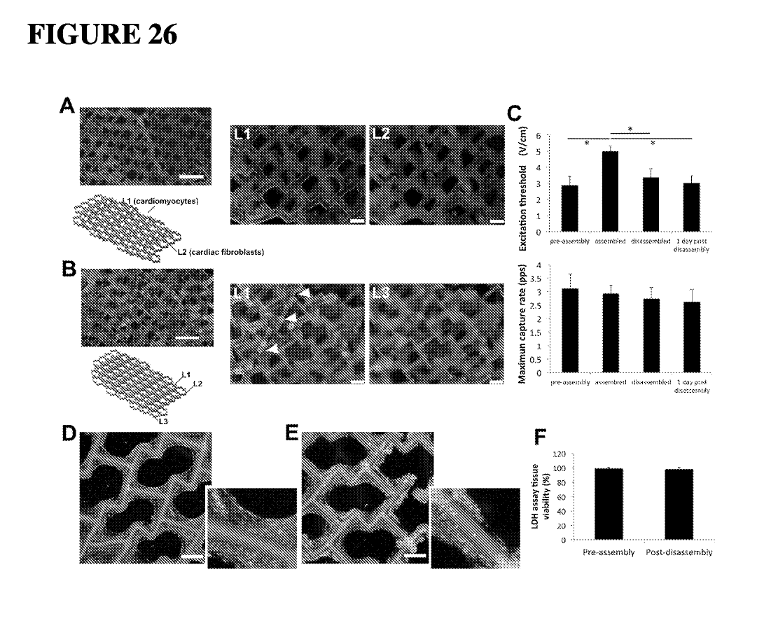

Individual tissues cultured in parallel were assembled simply by overlapping multiple tissues one on top of the other, allowing the hooks from one scaffold to grab onto the struts of the other scaffold (FIG. 26). This interlocking mechanism was achieved by a gentle compression of the two tissues together. Once affixed in place, each tissue could be separated only by specifically peeling one off another; handling or manipulating the entire multi-layer tissue did not disassemble the individual layers. During assembly, different cell types cultured on different scaffold meshes were positioned strategically to stack the tissues in the z-axis. To demonstrate this, we labeled rat cardiac FB and rat CMs, and affixed the layers together; instantaneously establishing co-culture conditions (FIG. 26A). The two-layer stack had a thickness of 580±5 μm, which was derived from the scaffold dimensions as well as based on the overlap configuration of two Interlocking tissue scaffold scaffolds. Additionally, three cardiac tissue meshes labeled with two different fluorescent cell trackers were locked into one tissue construct (FIG. 26B). The three-layer stack had a thickness of 712±7 μm. High magnification images show the hooks from the red tissue mesh penetrated through and locked onto the struts of the green tissue mesh on top (FIG. 26B).

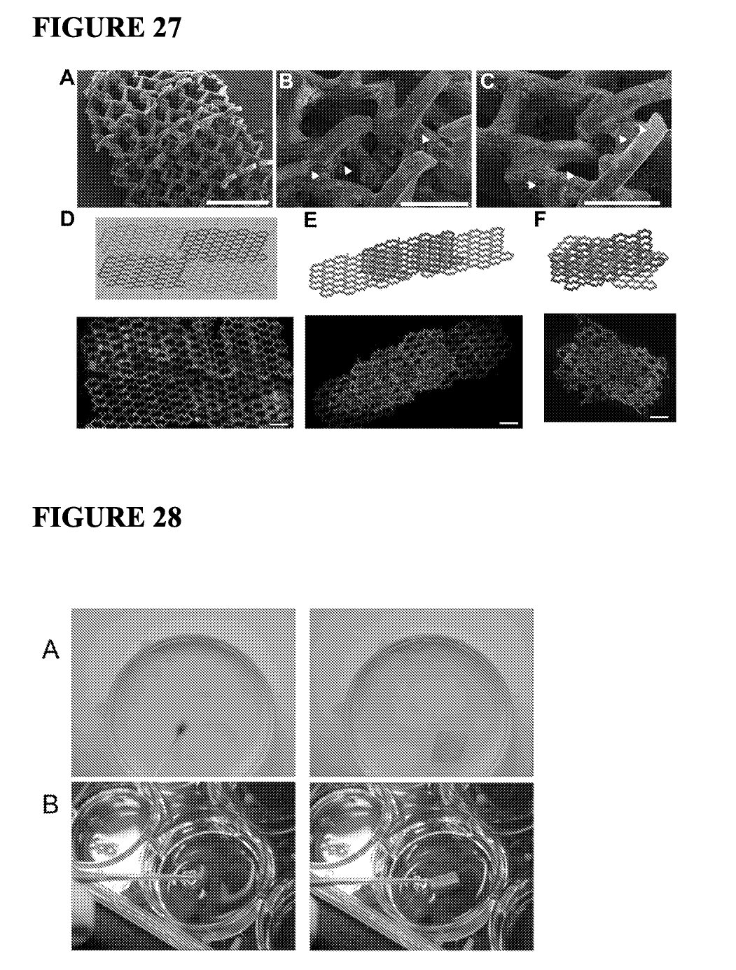

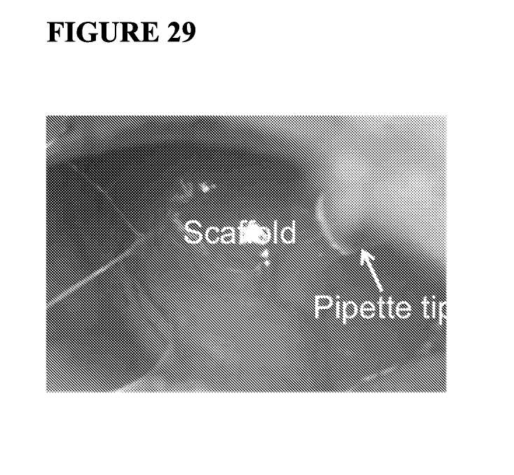

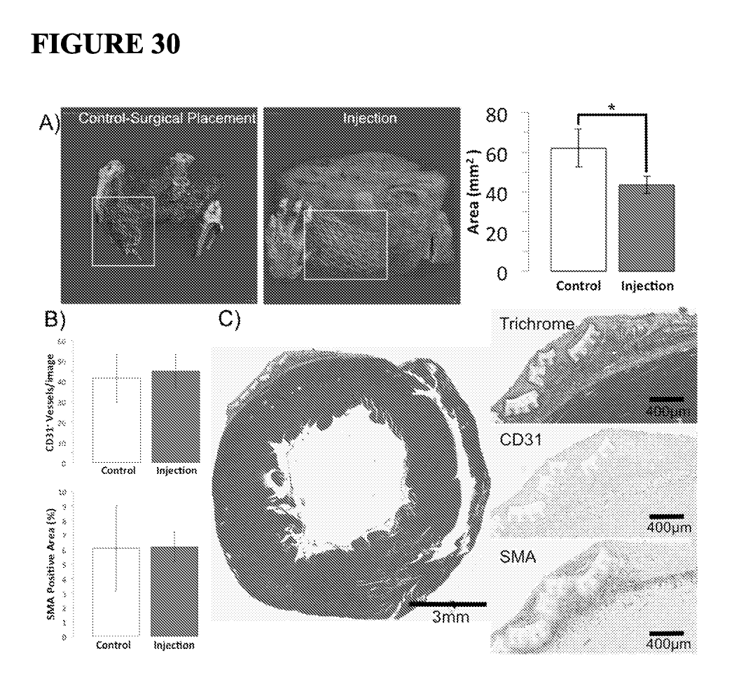

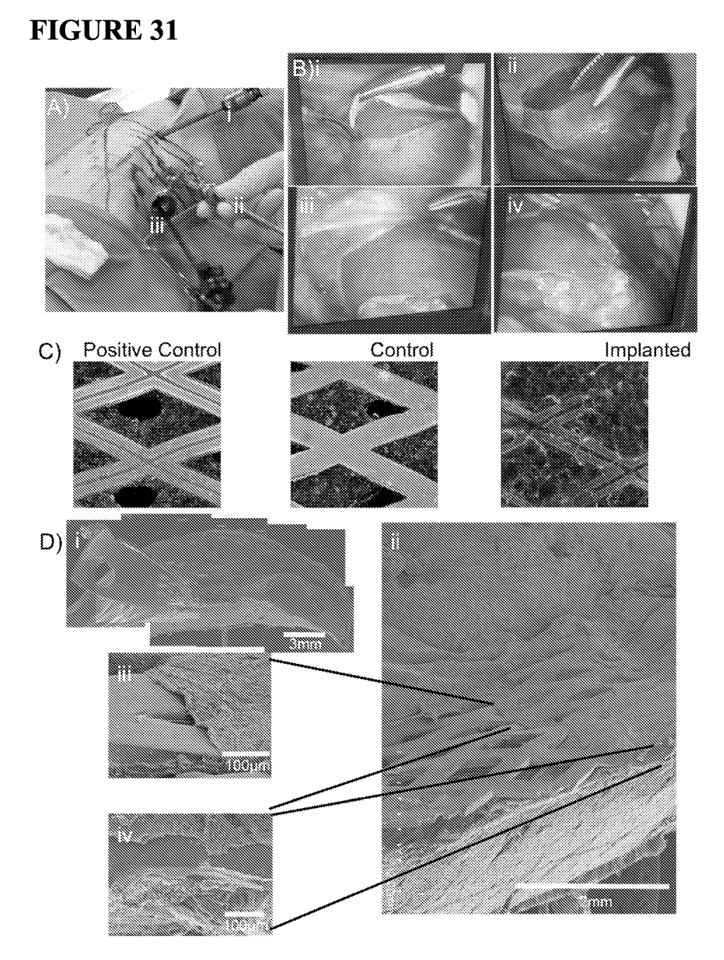

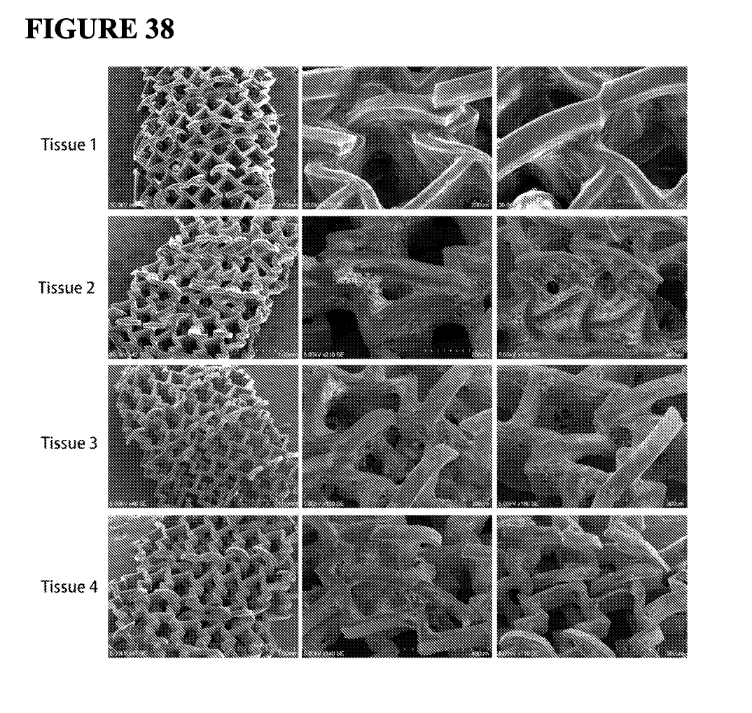

The electrical excitability properties of the cardiac tissues before assembly, after assembly (twolayer), after disassembly, and 1 day after disassembly were examined. Uniquely, the construct contracted synchronously under electrical field stimulation, immediately after assembly. We found that the excitation threshold (ET) increased slightly immediately after assembly and disassembly. However, the ET of the tissue decreased to its initial level 1 day after disassembly, likely due to tissue recovery (FIG. 26C). There were no changes in the maximum capture rate (MCR) of the tissues throughout the process (FIG. 26C). Viability staining indicated the absence of appreciable tissue damage upon layer assembly and disassembly, with majority of the cells staining positive for the viable dye CFDA (FIG. 26D,E). Lactate dehydrogenase (LDH) assay quantified the tissue viability at over 98% and showed no significant difference in cardiac tissue viability before assembly and after the two layer disassembly (FIG. 26F). Assembled tissues were cultivated for 3 days following assembly to demonstrate tissue integration between layers. SEM revealed that the hooks from the bottom tissue layer attaching onto the struts from the top tissue functioned as bridges allowing cell spreading and physical integration of the two layers (FIG. 27A-C, FIG. 38). Three days after assembly, tissues demonstrated synchronized spontaneous contractions indicating that the cell-cell connections between the layers have been established.

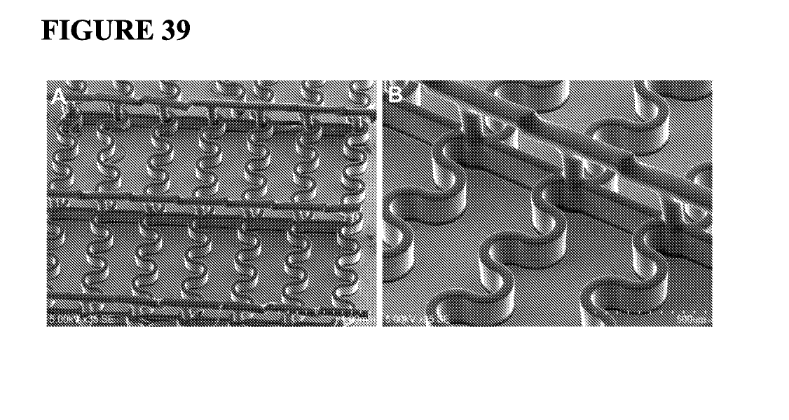

To demonstrate the versatility of co-culture patterning, we also assembled rat CM Interlocking tissue scaffold horizontally in a checkerboard pattern (FIG. 27D). The length of the scaffold network was extended by coupling three scaffolds in an overlapping end-to-end fashion (FIG. 27E). Two cardiac tissues were also stacked at 45 degrees demonstrating the feasibility of varying the cell orientation throughout the tissue depth (in z-direction) using this technology, to ultimately mimic the gradual change in myofiber orientation in the ventricular wall of the heart 18 (FIG. 27F). The design of Interlocking tissue scaffold is not limited to the accordion-mesh scaffold shape. Other designs with spring-like features (FIG. 39) were also produced. These designs could be used in future studies to enhance anisotropic tissue alignment and percent shortening at contraction. To accelerate the spatially organized tissue assembly and on-demand disassembly process, we introduce a new platform technology termed Interlocking tissue scaffold. We previously demonstrated cellular alignment and compaction along a simple surgical suture 19. Here we scaled the same concept to a more complex scaffold mesh. Cellular alignment is attributed mainly to the tension generated from the remodeling and alignment of the ECM against a template during the tissue formation process 20. In this study, the template was the primary scaffold mesh. The scaffold mesh was made of a synthetic elastic biodegradable polymer that provided mechanical stability and allowed manual handling and assembly. The scaffold also provided topographical cues for cellular orientation in the desired direction, as well as the anisotropic mechanical stiffness designed to mimic the native myocardium. Furthermore, by adding T-shaped hooks onto the scaffold mesh, we created an Interlocking tissue scaffold system allowing multiple cell types to be cultured individually and then assembled together vertically or horizontally to instantly establish a 3-D mosaic co-culture system, that could be disassembled on-demand.

While novel bioprinting techniques enable creation of tissues with a remarkable control over cell position, they do not allow for the release of cells or cell clusters without the destruction of the primary tissue structure. Additionally, re-assembly of the primary tissue units into a new structure is not possible and extensive time in culture is needed for cell orientation to be established in the gel-based systems 12,21. Elegant devices that pick, stack and perfuse selfassembled cell structures have been developed, but the mechanical stability of these stacked structures was achieved only after ˜48 hr when the cell-matrix remodeling resulted in the fusion of individual parts 22. Stackable polymeric scaffolds for scalable heart tissue engineering have been reported, however they are created by sequentially stacking and solvent bonding individual polymer layers followed by neonatal rat heart cell seeding and perfusion culture 23. Thus, the layers in the stacked device are not individually addressable and cannot be disassembled after the tissue is formed.

We adopted the general strategy of bottom-up tissue engineering, using microfabrication techniques to generate a miniaturized scaffold that can guide tissue remodeling followed by the assembly, with immediate functionality, into 3-D cardiac tissue while preserving the original tissue structure and topography. Injection molding of photo-crosslinkable POMaC enabled the fabrication of a variety of scaffold structures. POMaC was selected due to its biocompatibility as an implantable biomaterial, biodegradability and the potential to tune scaffold mechanical properties and processability in a wide-range through the dual (temperature and UV) crosslinking mechanism 17. The Young's modulus of the base material was initially 552 kPa, then 510 kPa upon 1 week in the presence of the cells and culture media (FIG. 32). The Young's modulus of the adult human myocardium was reported to be in the range of 200-500 kPa in the contracted state 24-27, thus the polymer has physiologically relevant bulk elasticity. Our novel microfabrication method allowed additional features to be patterned onto the 2-D mesh to form intricate 3-D structures, such as micro-hooks. The individual tissue meshes were assembled into functional 3-D tissue with the use of a hook and loop mechanism, thus creating 3-D functional tissues, e.g. a cardiac tissue capable of macroscopic contractions.

Although other cardiac tissue engineering techniques also provide tissues with small percent shortening at each beat 28, it is necessary to improve this functional parameter in order for the patches to become useful in the context of heart repair. If non-myocyte layers such as FBs or EC-tissue layers were used for 3-D assembly, the co-culture effect would take more time to become apparent, as these cells are not capable of contractile activity. In other co-culture methods that may include spatially defined cell positioning using hydrogels, as in bioprinting or soft lithography, CMs are rounded and do not form interconnected syncytium immediately after seeding. Thus, they are not capable of immediate contraction upon tissue fabrication and several days may be required for the cells to attach to the matrix, elongate and connect so that they can exhibit a synchronous contractile function. This tissue engineering strategy could also eliminate the need for a complicated perfusion bioreactor for in vitro culture of thick tissues. Each thin tissue mesh can be cultured separately without oxygen deficiencies and then assembled into a thick tissue construct prior to implantation. After assembly, the mass transfer of oxygen and nutrients could also be enhanced by the presence of void spaces within the tissue construct. An additional advantage of the Interlocking tissue scaffold 3-D scale-up is the fact that each layer is pre-fabricated and fully functional with a completed cell/gel remodeling process. This prevents a large-scale size change and delay in functionality that is usually observed with remodeling of 3D cell/hydrogel systems.

Co-culture is a tool used by cells biologists and tissue engineers for improving vascularization and cell survival by implementation of supporting signals that recapitulate an in vivo niche 6,29. Since a cell suspension can easily penetrate through the mesh structure, this allows ECs to coat around the tissue fibers on the Interlocking tissue scaffold scaffold mesh. ECs were demonstrated previously to support CM survival and viability in co-culture 30,31. In the native myocardium, ECs are organized in dense, branching tubular vascular structures with parallel capillaries in intimate contact with CM-bundles, such that each CM is positioned no more than 20 μm from the capillary 32,33. The described Interlocking tissue scaffold geometry does not capture the complexity of the native EC arrangement in a tubular branching vasculature, but it provides two important aspects of the native EC-CM configuration. First, EC coating in direct co-culture provides protection from shear, as coated CMs are not directly exposed to the flowing media. Second, ECs and CMs are in close physical proximity on Interlocking tissue scaffold, potentially enabling paracrine signaling between the two cell types, which usually decay rapidly as a function of spacing 34,35. The ability to coat the tissue with ECs can be beneficial when implanting the tissue. For example, the presence of tissue modules coated with ECs has been shown to enhance in vivo anastomosis and tissue survival 36. Modular tissue co-culture systems consisting of ECs and bone marrow-derived mesenchymal stem cells supported the survival and stable chimeric blood vessel anastomosis of ECs in vivo 37. Infiltration of cells from the host and implant integration could also be enhanced due to the macroporous tissue structure 38. Implanted cardiac cell sheets co-cultured with ECs were observed to have improved anastomosis and neovascularization 39.

The described platform technology also allows co-culture of multiple cell types in different tissue layers (such as CMs and cardiac FB). The importance of FB in cardiac tissue engineering has been well documented 40,41. For example, a non-myocyte preculture to support CMs resulted in improved cardiac organoid structure and function 35. Enhanced connexin 43 levels were achieved from the release of vascular endothelial growth factor secreted by precultured FB 42. The Interlocking tissue scaffold platform is compatible with sequential assembly of different cell types (e.g. cardiac FB followed by CMs) in a defined temporal sequence, thus potentially enabling preconditioning of the environment for the target cell type survival and optimized function. In the native myocardium, FBs are interspersed between CM 43. Alternating layers of CM and FB are used here to show the versatility of the technique and provide paracrine signaling. Stacking several CM layers has more physiological relevance than alternating CM/FB layers in the scaled-up tissue.

Examples of co-culture applications in tissue engineering for which the present system can be employed include heart, bone, cartilage, lung, kidney, liver, and nerve. The ability to coat the tissue in ECs can be beneficial when implanting tissue. For example, the presence of tissue modules coated with ECs has been shown to enhance in vivo anastomosis and tissue survival. Modular tissues co-culture systems consisting of bone marrow-derived mesenchymal stem cells supported the survival and degree of stable chimeric blood vessel anastomosis of ECs in vivo. Infiltration of cells from the host and implant integration could also be enhanced due to the macroporous tissue structure. Implanted cardiac cell sheets co-cultured with ECs were observed to have improved anastomosis and neovascularization. The importance of fibroblasts in cardiac tissue engineering has also been well documented. Iyer et al. demonstrated that having a non-myocyte (fibroblasts and ECs) preculture to support CMs resulted in improved cardiac organoid structure and function 28. Enhanced connexin levels were achieved from the release of vascular endothelial growth factor secreted by precultured fibroblasts and ECs. Injection molding of photocrosslinkable POMaC enables the fabrication of a variety of scaffold structures. The additional UV exposure also allows additional features to be patterned onto the 2-D mesh to form intricate 3-D structures.

POMaC material is well suited for cardiac tissue engineering because it is an elastomer that can be dynamically stretched and return to its original shape over cyclic loading; the honeycomb design further enhanced this property. The honeycomb design was previously investigated using poly(glycerol sebacate), and excimer laser microablation, a technique that cannot generate complex hook-shaped structures in the z-axis 8. The use of 3-D stamping together with injection molding was critical for the formation of T-shaped hooks here. Furthermore, recreating a graft that will integrate with the host myocardium and provide maximal therapeutic benefit requires structural reinforcement 44,45 and appropriate anisotropy 46,47 from the grafts.

The placement of an anisotropic patch onto an infracted heart has been shown to improve systolic function. Matching the anisotropic properties of the myocardium is important in ensuring that the presence of an epicardial patch does not impede heart contraction. There is also a benefit of mechanically reinforcing the heart wall. The interlocking tissue scaffold systems as described herein possesses anisotropic properties similar to the heart but remains soft allowing for deformation and mechanical transfer of the contraction.

The developed scaffold meshes possess mechanical properties (FIG. 24B) similar to the native rat neonatal myocardium (4.0 to 11.4 kPa) 48 but still allowing for deformation and mechanical transfer of the CM contraction. Each layer of the current Interlocking tissue scaffold design is thick compared to the individual laminar layers of the myocardium. Using soft lithography, we could create polymer layers as thin as 10-20 μm, however the mechanical stability of the overall structure would decrease, necessitating the use of polymer composition with a higher Young's modulus and denser spacing of the scaffold struts. We adopted the general strategy of using microfluidic technology or microfabrication techniques to generate miniaturized scaffold that can guide tissue remodeling followed by the assembly of these miniaturized scaffolds into functional 3-D tissue while preserving the original tissue topography. The creation of aligned 3-D cardiac tissue mesh capable of macroscopic contraction has been demonstrated. The tissue meshes were assembled into functional 3-D tissue with the use of a built-in hook and loop system mimicking the conventional Velcro® design. This tissue engineering strategy eliminates the need for a complicated perfusion bioreactor for culturing thick tissue. Each thin tissue mesh can be cultured separately without oxygen deficiency and then assembled into a full tissue construct prior to use. After assembly, the mass transfer of oxygen and nutrients is enhanced by the presence of void spaces within the tissue construct.

The stable polymeric structure makes Interlocking tissue scaffold less susceptible to damage due to physical handling during delivery. The meshes were easily handled with forceps and assembled into a desired pattern or arrangement. Compared to techniques such as cell-sheet technology 49, or collagen-based tissue mesh 21, Interlocking tissue scaffold maintained its own structure without external substrate support, and it was flexible enough to regain its shape after deformation. Interlocking tissue scaffold is a platform technology based on a biocompatible, implantable and biodegradable polymer, that can easily be transferred, in future studies, to cell co-culture in multiple settings (e.g. for skin or liver tissue engineering, etc.).

The ability to dynamically control both spatial and temporal culture parameters, enables the potential use of this technology in cell differentiation, e.g. timed application of growth factors and selective, timed, cell-cell contact. Alternatively, individual cell layers could be separately treated with different survival factors prior to assembly of the tissue for implantation to maximize its ability to survive in inflammatory or hypoxic environments. Ability to disassemble the tissues on-demand may provide a tool for spatially defined follow-up studies, e.g. to determine how cell viability, metabolism or gene expression vary as a function of thickness in different culture or implantation conditions. These individual layers from different tissue depths and various cultivation conditions could then be strategically re-combined to study the possibility that cells retain memory of their previous environment, with a view of optimizing cell survival and differentiation protocols for in vitro and in vivo studies.

Exemplary Materials and Methods

POMaC pre-polymer synthesis. The interlocking tissue scaffold was made out of an elastic, biodegradable, dual cross-linkable (heat and UV) elastomer (poly(octamethylene maleate (anhydride) citrate), POMaC) as synthesized previously 17. Briefly, 1,8-octanediol, maleic anhydride, and citric acid were added to a 250 mL triple-neck flask at a molar ratio of 5:1:4, respectively. The reaction vessel was heated to 160° C. and stirred until a clear solution was formed before subsequently decreasing the temperature to 140° C. for 3 hours under nitrogen purge. Then, POMaC pre-polymer was dissolved in ethanol and purified by drop-precipitation into deionized water followed by 3 days of lyophilization. The purified POMaC pre-polymer solution was then mixed with poly(ethylene glycol) dimethyl ether (PEGDM, Mw˜500, Sigma) at 60 wt % and 5 wt % UV initiator (2-hydroxy-1-[4(hydroxyethoxy)phenyl]-2-methyl-1 propanone, Irgacure 2959). Poly(ethylene glycol) was used as a porogen to reduce the viscosity of the pre-polymer solution during injection into the mold. The porogen was leached out in phosphate buffered saline (PBS) after scaffold fabrication. POMaC degradation. Pre-POMaC strips (1.5 mm×0.5 mm×10 mm) were UV (365 nm) exposed with 8100 mJ/cm2. The strips were weighted in sets of 10 to determine initial mass. They were then soaked in PBS for 2 hr followed by 70% ethanol overnight and additional two washes in PBS. The strips were then placed into transwell inserts (one strip/well) of a 24 well plate, with rat CMs seeded at the bottom and cultivated in the CM culture media. Strips were collected at 1 day and 14 days, washed twice in deionized distilled water and lyophilized for three days. Final mass was recorded and reported at each time point as percentage of mass lost compared to the immediately fabricated scaffold (day 0).

Bioadhesive pre-polymer synthesis. A polycondensation of polyethylene glycol (PEG, Mn 400 g·mol), citric acid, maleic anhydride, and dopamine was carried out in a molar ratio of about 1:0.66:0.44:0.5 respectively. Citric acid, maleic anhydride, and PEG were added to a 250 mL triple neck flask. The reaction vessel under nitrogen purge was heated to about 160° C. and stirred until a clear solution formed. After about 3 minutes, dopamine was added and the temperature was decreased to about 140° C. The reaction was carried out to completion for about 3 days under nitrogen purge. The photocrosslinkable injectable citric acid bioadhesive (PICAB) pre-polymer was dissolved in about 50 mL of DI H₂O and dialyzed for about 1 day followed by snap-freezing and about 3 days of lyophilization. The purified PICAB pre-polymer solution was then mixed with the porogen, poly(ethylene glycol) dimethyl ether (PEGDM, Mw˜500, Sigma) at about 60 wt % and about 5 wt % UV initiator 2-hydroxy-1-[4(hydroxyethoxy)phenyl]-2-methyl-1 propanone (Irgacure 2959). The final mixture was stored under nitrogen and kept in the dark at about 4° C.

Polymer Characterization. An ampoule containing about 1% (wt/v) solution of the polymer dissolved in dimethyl sufloxide (DMSO) was placed in a Proton nuclear magnetic resonance (1H-NMR) machine (Varian Unity 500, Nuclear Magnetic Resonance facility, Department of Chemistry, University of Toronto) to confirm the polymer composition. ATR-FTIR was used to confirm the presence of functional groups.

Scaffold Fabrication. The device was fabricated using standard SU-8 photolithography techniques as previously described 50. Briefly, SU-8 photoresist was spin-coated on silicon wafers according to manufactures' guidelines. SU-8 photoresist was exposed to 365 nm UV using a mask aligner (Q2001, Quintel Co., San Jose, Calif.) through transparency masks with features of desired shape. The multi-layered device required proper alignment between the features on the first and second layers before exposure. The nominal width of the mesh and the hooks was 50 μm and 100 μm respectively while the height of the bottom layer (mesh), the middle layer (post), and the top layer (hooks) were 132±5 μm, 263±5 μm, 53±1 μm, respectively. Finally, the master mold was submersed in SU-8 developer solution until all the unexposed photoresist was dissolved from the surface. A negative of the mold was made by pouring poly(dimethylsiloxane) (PDMS) elastomer with a curing agent (17.5:1 ratio) and curing at room temperature for 3 days (FIG. 23B). The PDMS molds were then capped with either a glass slide or a flat sheet of PDMS to form a closed network of channels (FIG. 23B). The POMaC pre-polymer/porogen/UV initiator mixture was then slowly injected through the mold at the inlet and left overnight to allow trapped air bubbles to dissipate. The PDMS molds were exposed at 2400 mJ/cm2 (the exact UV exposure energy was fine-tuned for each batch of pre-polymer solution) followed by peeling PDMS molds from either the glass or the PDMS cap. A PDMS mold and a glass slide with the scaffold were aligned and pressed together and exposed at 2400 mJ/cm2 to covalently bond the two layers together (FIG. 23B). The connections between each T-shaped hook on the scaffold were then cleaved with Vannas spring scissors (Fine Science Tools) (FIG. 23B). The T scaffold was then removed from the substrates and placed in PBS (FIG. 23B). Individually cultured tissues were then assembled with fine tweezers by manual manipulation at the specified time point.

Scaffold structure characterization. Scanning electron microscopy (SEM) was used to assess scaffold and tissue structure using a Hitachi SEM S-3400 in secondary electron mode at the Microscopy Imaging Laboratory, Faculty of Medicine, University of Toronto. Prior to imaging, the tissues were fixed in a 1% glutaraldehyde/4% paraformaldehyde mix overnight at 4° C., washed in PBS and dehydrated in sequential washes of 50%, 70%, 95% and 100% ethanol, followed by critical point drying. Optical prolifometry (Bruker Contour GT-K, 10× parfocal objective) was used to assess the height of the scaffold features.

Mechanical characterization. The mechanical properties of the scaffold were measured in PBS with a Myograph (Kent Scientific) in long edge and the short edge direction. The slope of the uniaxial tensile stress-strain curve from the first 15% strain was used to approximate the physiological regime and the linear portion was used to calculate the effective elasticity as described 8,51-53. To determine the linear region the entire data set was fitted using a least-squares regression followed by repeatedly dropping the lowest strain data point until the maximum R2 value was achieved. The anisotropy ratio was determined by dividing the effective elasticity in the long-edge direction with the effective elasticity in the short-edge direction. The initial scaffold length and width was measured with a caliper for stress calculations. Tensile tests were also conducted on samples of crosslinked POMaC strips, prepared in the mold with dimensions 1.5 mm×0.5 mm×10 mm, to determine the mechanical properties of the bulk material over time. Strips were prepared and treated as described in POMaC degradation. Strips were collected and tested at 1 day and 7 days after exposure to cells and culture media. Tensile testing was performed by pulling POMaC strips, submersed in PBS, along the length of the sample with a Myograph (Kent Scientific). Stress and strain relationships were plotted and the Young's Modulus was taken from the slope of the linear portion of the curve.

In vitro injections. To assess how well each design could be delivered in a minimally invasive manner, 1 cm2 scaffold were submersed in PBS in a glass Pasteur pipette (˜1 mm inner diameter) and a 50 mL Silicone pipette bulb was compressed to eject the PBS/scaffold into a dish containing PBS. The ability for the scaffold to be injected (without damage) and regain its initial shape were recorded over multiple injections (n≧6).

Pull-off force measurement. The pull-off force of the scaffolds was measured in PBS with Myograph (Kent Scientific). One scaffold was first glued to the bottom of a Petri-dish or pinned down with two micro-pins to a PDMS base in a Petri dish. If the scaffold was glued to the bottom of the Petri-dish, the second scaffold placed on the top was overlaid by 100% with the bottom scaffold. In the case of scaffolds cultivated with cells, glue could not be applied and they were pinned down to the PDMS coated Petri-dish. Then upper scaffold of tissue was applied in the partly offset configuration, in order not to interfere with the pin. A micro-needle connected to the 2-gram force transducer was hooked onto the outer right strut of the top scaffold and it was pulled rightwards with a micromanipulator until the top scaffold layer was completely released. The force generated during the process was recorded and the maximum peak force prior to release was the pull-off force. The last data point collected after complete scaffold release was used as the baseline for force measurement. The nominal area of the scaffold (2.5×5 mm) was used in calculation.

In vivo work. Preliminary microCT images have been captured of the scaffold in vivo. Porogen leached scaffolds were soaked in barium sulphate (x-ray contrast agent) solution for 1 hour, rinsed in PBS, and placed with tweezers subcutaneously into a euthanized mouse. 30G needles were placed 5 mm above and below the scaffold to act as a guide for trouble shooting and location purposes.

Neonatal rat heart cell isolation. Neonatal rat heart tissue was digested as described previously. Briefly, neonatal (1-2 day old) Sprague-Dawley rats were first euthanized and hearts were excised and placed in ice-cold Ca2+ and Mg2+ free Hank's balanced salt solution (HBSS) (Gibco, Canada). Before quartering the heart the aortic and vena cava structure were removed. Heart sections were rinsed twice in ice-cold HBSS and digested in an about 0.06% (w/v) solution of trypsin (Sigma, Canada) in HBSS overnight at about 4° C. Collagenase II (Worthington, USA 220 units/mL) in HBSS was used to further digest the heart tissue at about 37° C. in a series of five 4-8 min digestions. After the collagenase digestion, cells were pre-plated for about 40 mins. The non-adherent cells were used as enriched cardiomyocyte population. The purified cardiac FB population was obtained from the adherent cells. Cardiac FB were cultured and passaged once before use.

Cell seeding and culture. Cell-hydrogel preparation was carried out as similarly described by Nunes et a154. Briefly, a desired number of freshly isolated cardiomyocytes or cardiac FB were first pelleted and suspended in a liquid Matrigel solution at a ratio of 1 million cells to 1 μL Matrigel solution. Typically a 20 μL of cell/Matrigel mixture was made at a time. Prior to cell seeding the scaffolds were coated in a 0.2 wt % gelatin solution in PBS at 37° C. for 4 hours to facilitate cell attachment. 2 μL of cell suspension was pipetted onto the scaffold to cover the scaffold with cells in a 6 well cell culture plate (FIG. 23C). Excessive gel was removed until only a thin layer of gel/cell suspension covered the scaffold. The plate was then placed in an incubator for 4-6 min to allow the Matrigel mixture to partially gel. Pre-warmed culture medium was then added and a cell scraper was used to gently scrape the scaffold off the bottom of the plate. After the scaffold (initially fully covered with cells) was lifted, holes were then formed at the center of each honeycomb of the scaffold mesh due to the lack of structural support (FIG. 23C). Cells located near the scaffold struts remained on the scaffold. Media was changed once every 48 hours. The tissue constructs were cultured for one week prior to assembling and imaging (FIG. 23C). Rat cardiomyocytes and cardiac FB were cultured in Dulbecco's Modified Eagle Medium (DMEM, Gibco, Canada) containing 4.5 g/L glucose, 10% (v/v) fetal bovine serum (FBS, Gibco, Canada), 1% (v/v) HEPES (100 units/mL, Gibco Canada) and 1% (v/v) penicillin-streptomycin (100 mg/mL, Gibco, Canada).

Endothelial cell coating. Human umbilical vein endothelial cells (HUVECs) were purchased from Lonza and cultured with endothelial growth medium (EGM-2, Lonza) according to the manufacturer's instructions. Passage 3-5 HUVECs were used for all experiments. To coat the tissue meshes with endothelial cells, the tissues were immersed in 200 μL endothelial cells suspension with 50 million cells/mL for 2 hr to allow endothelial cell attachment. The cell suspension was gently disturbed once every 30 min. 2 mL of culture media was then added and tissue was incubated overnight to allow endothelial cells proliferation. EGM-2 was used for co-culture conditions with rat CMs and HUVECs. Co-cultured constructs were cultured for 2 days to allow for a confluent EC layer to form prior to imaging. Interlocking tissue scaffold scaffolds coated with ECs or without ECs were also cultured in 25 mL EGM-2 media in 125 mL shaker flasks orbiting at 160 RPM for an additional 3 days prior to imaging.

Functional characterization of cardiac interlocking tissue scaffold assembly. Assessment of the contractile behavior of the cardiac sheets was measured using an S48 Grass Stimulator (Grass Technologies/Astro-Med Inc) as described previously 28,35. At day 7 post-seeding cardiac sheets were placed into stimulation chambers and stimulated with a biphasic square 2 ms pulse duration at 1 Hz. The excitation threshold (ET, V/cm) was determined by increasing the output from 0 V at 0.1 V increments until synchronous cardiac sheet contraction was observed in unison with the stimulator output. The maximum capture rate (MCR, Hz) was determined by setting the output voltage to double the ET and increasing the frequency of stimulation in 0.1 pulse per second (pps) increments until the cardiac sheet beating could not keep pace with the stimulator output. Video analysis was performed in ImageJ (version 1.47v) first by thresholding the video followed by outlining the scaffold to acquire a single tracer outline of the struts of the scaffold mesh. Using this outline, the change in the area of the honeycomb mesh was tracked overtime. The degree of scaffold deformation was derived from the decrease in the honeycomb size due to tissue contraction. The shortening of the long and short axis was measured using image analysis to detect the percentage shortening. Cell orientation on the tissues was characterized with Image J from the confocal images of the tissues stained with 4′,6-diamidino-2-phenylindole (DAPI). Each section of the confocal Z-stack was processed separately. The cell nuclei were selected from the images with thresholding and then turned into binary images. Nuclei that appeared merged together or out of focus were eliminated. The orientation of each selected nucleus was then plotted in MATLAB with the Quiver function. Orientation of the scaffold struts was quantified using an Image J plug-in, OrientationJ55, from the same confocal Z-stack images. The Erode function in Image J was used to filter out the small cell nuclei and leave out only the scaffold struts. The images were then processed and plotted with OrientationJ. To stimulate cardiac tissues with drugs, epinephrine was first dissolved in HCl (12.1N) and was then diluted to 0.3 μM in cardiomyocyte culture media. Drug solution was applied to spontaneously beating tissue and the response of the tissue was recorded.

Contractility Function. Assessment of the contractile behavior of the cardiac sheets was measured using a S48 Grass Stimulator (Grass Technologies/Astro-Med Inc) as described previously. For stimulated samples, cardiac sheets were placed into stimulation chambers and stimulated with a biphasic square pulse for about 2 ms at about 1 Hz and about 3 V/cm on day four days post-seeding. The tissues were cultured for two weeks. The excitation threshold (ET, V/cm) was determined by increasing the output from 0 V at about 0.1 V increments until synchronous cardiac sheet contraction was observed in unison with the stimulator output. The maximum capture rate (MCR, Hz) was determined by setting the output voltage to double the ET and increasing the frequency of stimulation in about 0.1 V increments until the cardiac sheet beating could not keep pace with the stimulator output. ET and MCR data were gathered immediately before and after injections.

Immuno-fluorescent staining. Immuno-fluorescent staining was performed to assess the morphology of the cultivated tissues. The tissues were first fixed in 4% (w/v) paraformaldehyde in PBS for 15 min at room temperature. Then, the cells were permeated and blocked in 5% FBS and 0.25% Triton X100 in PBS for 1 hour. Next, the tissues were incubated in primary antibody against sarcomeric α-actinin (Mouse, 1:200, Abcam, ab9465), overnight at 4° C., followed by incubation with a secondary antibody, Alexa 488 conjugated anti-mouse IgG (1:200, Life Technologies, A21202) and a phalliodin 66 conjugated anti-F-actin (1:300, Life Technologies, A22285). Tissues were then washed and imaged with confocal microscopy (Olympus FV5-PSU confocal with IX70 microscope, Canada). To visualize the endothelialized coating, the tissues were fixed in 4% PFA and blocked in 5% FBS for 1 hour. Then, the scaffolds were incubated in primary antibody, CD31 (Mouse, 1:200 dilution, MAB2148), followed by incubation with secondary antibody; Alexa 647 conjugated anti-mouse IgG (1:200 dilution, Sigma). To visualize the tissue in the co-culture experiments, prior to assembly, each tissue was incubated in either carboxyfluorescein diacetate (CFDA-SE, 1:1000, Life Technologies, C1157) or CellTracker Red (CMPTX, 5 μM, Life Technologies, C34552) in PBS at 37° C. for 30 min. Assembled tissue constructs were image immediately after assembly. DAPI was used to visualize cell nuclei.

Tissue viability and LDH Assay. Tissue viability was visualized with CFDA-SE (1:1000, Life Technologies, C1157) and propidium iodide (PI, Life Technologies, P3566) in PBS as shown previously56. Cell death analysis was performed on culture media collected from tissues pre-assembly and post disassembly using an LDH Cytotoxicity Assay Kit (Cayman Chemical Company) as per instructions given by the manufacturers. Tissues were also lysed with 0.1% Triton X100 to release all the LDH from the cells in a tissue construct as a baseline for 0% viability. The percentage of dead cells was determined by dividing LDH measured in the media, by total LDH released upon cell lysis. To obtain the percentage of viable cells plotted in the graph, the percentage of dead cells was subtracted from 100%.

Statistical analysis. Error bars in figures represent standard deviation. Statistical analysis was performed using SigmaPlot 12. Normality and equality of variance for the data was tested and an appropriate statistical test was used. Statistical analysis was determined using a Student's t-test, performed with one-way ANOVA followed by Tukey-Kramer test, or Mann-Whitney Rank Sum test. A p-value of less than 0.05 was considered significant. A minimum of 3 samples were used per data point, as indicated in the figure captions.

In certain aspects the description provides a shape-memory polymer fiber tissue scaffold comprising micro- or nano-sized elastomeric fibers or a combination thereof, wherein the fibers are arranged into a reversibly deformable design or configuration. In any of the aspects or embodiments described herein, the deformable design or configuration comprises a rhomboidal or diamond-shaped geometrical configuration. In any of the aspects or embodiments described herein, the scaffold is seeded with a precursor or progenitor cell, e.g., a cardiac myocyte. In any of the aspects or embodiments described herein, an electrical stimulation may be delivered across the scaffold. In any of the aspects or embodiments described herein, at least one fiber surface comprises a channel that runs along the length of the fiber.

In any of the aspects or embodiments described herein, the scaffold comprises an array of micro-hooks extending from a surface of the fibers. In any of the aspects or embodiments described herein, the micro-hooks are formed of a polymer fiber post extending approximately vertically from the plane of the polymer tissue scaffold, and including a polymer fiber cross-bar attached to the post. In any of the aspects or embodiments described herein, the micro-hook has a T-shape.

In any of the aspects or embodiments described herein, the polymer fibers are produced by reacting 1,8-octanediol, maleic anhydride, and an acid. In any of the aspects or embodiments described herein, the acid is at least one of 1,2,4-butanetricarboxylate, citric acid or a combination of both.

In a further aspect, the description provides a tissue scaffold system comprising an interlocking polymer fiber layer comprising micro- or nano-sized elastomeric fibers or a combination thereof, wherein the fiber layer has a top surface and a bottom surface, and includes an array of micro-hooks extending from at least one surface. In any of the aspects or embodiments, the system further comprises a polymer fiber layer in apposition with the interlocking polymer layer, wherein the polymer fiber layer includes loops or voids therethrough that are of sufficient size to allow intercalation or engagement with the micro-hooks of the first polymer fiber mesh layer when the layers are placed in apposition. In any of the aspects or embodiments, the layers are reversibly secured when placed in apposition.

In any of the aspects or embodiments described herein, the tissue scaffold comprises a plurality of interlocking polymer fiber layers aligned vertically.

In any of the aspects or embodiments described herein, a polymer fiber layer is inserted between each interlocking polymer fiber layer.

In any of the aspects or embodiments described herein, the tissue scaffold is seeded with a precursor or progenitor cell, e.g., a cardiac myocyte.

In any of the aspects or embodiments described herein, an electrical field is delivered across the scaffold.

The description provides methods of treating or ameliorating a disease or condition comprising providing a tissue scaffold or tissue scaffold system of the aspects or embodiments described herein, seeding and growing a cell or tissue on the scaffold, optionally implanting or contacting the scaffold at a site in or on a subject in need thereof, wherein the tissue scaffold is effective for treating or ameliorating at least one symptom of the disease or condition.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the invention described herein. Such equivalents are intended to be encompassed by the following claims. It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. A shape-memory polymer fiber tissue scaffold comprising micro- or nano-sized elastomeric fibers or a combination thereof, wherein the fibers are arranged into a reversibly deformable design or configuration.

2. The tissue scaffold of claim 1, wherein the deformable design or configuration comprises a rhomboidal or diamond-shaped geometrical configuration.

3. The tissue scaffold of claim 1, wherein the scaffold is seeded with a precursor or progenitor cell.

4. The tissue scaffold of claim 1, wherein the scaffold is seeded with a cardiac myocyte.

5. The tissue scaffold of claim 3, wherein electrical stimulation is delivered across the scaffold.

6. The tissue scaffold of claim 1, wherein at least one fiber surface comprises a channel that runs along the length of the fiber.

7. This tissue scaffold of claim 1, wherein the scaffold comprises an array of micro-hooks extending from a surface of the fibers.

8. The tissue scaffold of claim 7, wherein the micro-hooks are formed of a polymer fiber post extending approximately vertically from the plane of the polymer tissue scaffold, and including a polymer fiber cross-bar attached to the post.

9. The tissue scaffold of claim 8, wherein the micro-hook has a T-shape.

10. The tissue scaffold of any of claims 1-9, wherein the polymer fibers are produced by reacting 1,8-octanediol, maleic anhydride, and an acid.

11. The tissue scaffold of claim 10, wherein the acid is at least one of 1,2,4-butanetricarboxylate, citric acid or a combination of both.

12. A tissue scaffold system comprising an interlocking polymer fiber layer comprising micro- or nano-sized elastomeric fibers or a combination thereof, wherein the fiber layer has a top surface and a bottom surface, and includes an array of micro-hooks extending from at least one surface.

13. The tissue scaffold system of claim 12, wherein the system further comprises a polymer fiber layer in apposition with the interlocking polymer layer, wherein the polymer fiber layer includes loops or voids therethrough that are of sufficient size to allow intercalation or engagement with the micro-hooks of the first polymer fiber mesh layer when the layers are placed in apposition.

14. The tissue scaffold system of claim 13, wherein the layers are reversibly secured when placed in apposition.

15. The tissue scaffold system of claim 12, wherein the scaffold comprises a plurality of interlocking polymer fiber layers aligned vertically.

16. The tissue scaffold system of claim 15, wherein a polymer fiber layer is inserted between each interlocking polymer fiber layer.

17. The tissue scaffold system of claim 12, wherein the scaffold is seeded with a precursor or progenitor cell.

18. The tissue scaffold system of claim 17, wherein the scaffold is seeded with a cardiac myocyte.

19. The tissue scaffold system of claim 18, wherein an electrical field is delivered across the scaffold.

20. The tissue scaffold system of any of claims 12-19, wherein the polymer fibers are produced by reacting 1,8-octanediol, maleic anhydride, and an acid.

21. The tissue scaffold system of claim 20, wherein the acid is at least one of 1,2,4-butanetricarboxylate, citric acid or a combination of both.

22. A method of treating a disease or condition comprising providing a tissue scaffold or tissue scaffold system of any of claims 1-20, seeding and growing a cell or tissue on the scaffold, optionally implanting or contacting the scaffold at a site in or on a subject in need thereof, wherein the tissue scaffold is effective for treating or ameliorating at least one symptom of the disease or condition.
